(19) United States
(12) Patent Application Publication
(10) Pub. No.: US 2025/0282846 A1
(43) Pub. Date: Sep. 11, 2025

(54) Compositions and Methods for Treating Cancer and Viral Infections

(71) Applicant: The University of Chicago, Chicago, IL (US)

(72) Inventor: Juan Mendoza, Chicago, IL (US)

(21) Appl. No.: 18/859,874

(22) PCT Filed: Apr. 25, 2023
(86) PCT No.: PCT/US2023/066209

§ 371 Date: Oct. 24, 2024

Related U.S. Application Data
(60) Provisional application No. 63/334,610, filed on Apr. 25, 2022.
(60) Provisional application No. 63/377,309, filed on Sep. 27, 2022.

(51) Int. Cl. C07K 14/715; A61K 38/00; A61P 35/00

(52) U.S. Cl. CPC C07K 14/7156; A61P 35/00; C07K 14/7155; A61K 38/00

(57) ABSTRACT

This disclosure relates to compositions and methods for treating cancer and viral infections.

### CROSS-REFERENCE TO RELATED APPLICATIONS

This application claims priority to U.S. Provisional Patent Application Ser. No. 63/334,610, filed Apr. 25, 2022, entitled, “Compositions and Methods for Treating Cancer and Viral Infections,” which is incorporated by reference herein in its entirety. This application also claims priority to U.S. Provisional Patent Application Ser. No. 63/377,309, filed Sep. 27, 2022, entitled “Compositions and Methods for Treating Cancer and Viral Infections,” which is incorporated by reference herein in its entirety.

### BACKGROUND OF THE DISCLOSURE

### Field of Invention

This disclosure relates to compositions and methods for treating cancer and viral infections.

### SEQUENCE LISTING

A computer readable form of the Sequence Listing is filed with this application by electronic submission and is incorporated into this application by reference in its entirety. The Sequence Listing is contained in the file created on Apr. 25, 2023, having the file name “22-0712-WO.xml” and is 14 kb in size.

### Technical Background

There is currently a tremendous effort in Pharma to develop treatments for cancer and a number of viral infections including but not limited to respiratory infections such as COVID19 and influenza, and other infections such as Hepatitis B, Hepatitis B/Hepatatis D co-infections, and Norovirus. Patients with persistent Hep B, Hep B/D co-infections have higher risks for development of liver cancers.

Type I Interferons (IFNs) are potent drugs used in a number of viral infections and as anticancer agents. Limiting the use of this class of Type I IFNs are the observed severe side-effects. Many patients are unable to complete the course of treatment, thus, limiting the potential for controlling or curing the infection.

In Phase Ia clinical trials of Peg-IFNL1 for the treatment of Hepatitis C, a Type III IFN demonstrated lower incidences of side-effects compared to Type I IFN treatment. However, Type III IFNs were found to be limited in their anti-viral efficacy. The limited efficacy has contributed to the less than desired clinical benefits against HCV and Hep D and SARS-COV2 (Jagannathan et al., Peginterferon Lambda-1a for treatment of outpatients with uncomplicated COVID-19: a randomized placebo-controlled trial. Nat Commun 12, 1967 (2021)).

Therefore, there is a need to further develop approaches to treat cancer and viral infections that can take advantage of interferon-associated treatment efficacy without the detrimental side-effects.

### SUMMARY OF THE DISCLOSURE

This disclosure describes compositions and methods for treating cancer and viral infections.

In a first aspect, the present disclosure provides an engineered cytokine receptor, wherein the receptor has an altered geometry that potentiates signaling when the receptor is bound by a ligand.

In a second aspect, the present disclosure provides a method of treating a patient for cancer and/or a viral infection, comprising: a) administering to the patient a therapeutically effective amount of a therapeutic agent that potentiates signaling through a cytokine receptor complex to provide a therapeutic effect; and b) treating the cancer and/or viral infection.

In one embodiment of the first aspect, the therapeutic agent is administered orally, intravenously, intraperitoneally, subcutaneously, or intratumorally.

In one embodiment of the second aspect, the cancer is melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplastic syndrome, multiple myeloma, transitional cell carcinoma, neuroblastoma, plasma cell neoplasms, Wilm's tumor, glioblastoma, retinoblastoma, or hepatocellular carcinoma.

In one embodiment of the second aspect, the virus causing the viral infection is HBV, HBV/HDV co-infection, Norovirus, Influenza, and/or SARS-COV2.

In a third aspect, the present disclosure provides an engineered cytokine receptor complex, comprising: a) one or more cytokine receptors; b) a transmembrane domain that comprises one or more mutations that promote heterodimerization of the receptor; and c) optionally, one or more high-affinity ligands bound to the one or more cytokine receptors.

In a fourth aspect, the present disclosure provides a cell, comprising the engineered cytokine receptor of the third aspect.

In one embodiment of the fourth aspect, the one or more cytokine receptors elicits signaling through a Janus kinase/Signal Transducer and Activator of Transcription (JAK/STAT) pathway in the cell.

In one embodiment of the fourth aspect, the engineered cytokine receptor is a Type III interferon receptor.

In one embodiment of the fourth aspect, the engineered cytokine receptor is IFNλR1, IL10Rβ, or IFNλ3 H11.

In one embodiment of the fourth aspect, the engineered cytokine receptor comprises one or more mutations compared to the corresponding wildtype receptor.

In one embodiment of the fourth aspect, the one or more mutations introduces at least one alanine into alpha-helical transmembrane domain of the receptor.

In one embodiment of the fourth aspect, the one or more mutations renders the receptor able to heterodimerize through the transmembrane domain.

In one embodiment of the fourth aspect, the one or more mutations renders the transmembrane able to structurally twist such that janus kinases associated with the transmembrane domain are oriented to permit cross phosphorylation and activation.

In one embodiment of the fourth aspect, the rotation decreases the distance between the kinase domains of janus kinases within the signaling complex, facilitating a more efficient transphosphorylation that leads to enhanced biological activities for Type III IFNs.

In one embodiment of the fourth aspect, the cell is an immune cell

In one embodiment of the fourth aspect, the cell is in vitro.

In one embodiment of the fourth aspect, the cell is in vivo.

In one embodiment of the fourth aspect, the cell is in vivo in a mammal.

In one embodiment of the fourth aspect, the mammal is a human.

In one embodiment of the fourth aspect, the human is in need of therapy and a therapeutically effective amount of cells is provided to the human.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIG. 1 provides an overview of Type I and Type III interferon cytokine-mediated receptor dimerization and downstream signaling pathways.

FIG. 2 depicts the relative positioning of proximal JAKs (left panel) and a schematic diagram (right panel) of alanine insertion mutagenesis (depicted sequences are SEQ ID NOS: 1-5, from top to bottom, respectively) of the IFN-λR1 transmembrane domain (top) and α-helical wheel projections of the register rotations introduced by addition of each alanine residue (bottom).

FIG. 3 is a schematic diagram that shows the proposed positioning of C-terminal kinase domain of JAK1 relative to its N-terminal FERM SH2 domain when viewed down the axis of rotation. When 2 alanine residues are inserted in the transmembrane region of IFN-λR1 receptor, the near 180-degree rotation to the intracellularly associated JAK1 orients the kinase domains of JAK1 and TYK2 in a front-to-back manner, posing a physical barrier to transphosphorylation. On the contrary, the 327-degree rotation afforded by 3 alanine insertion decreases the distance between the kinase domains of JAK1 and TYK2 within the signaling complex, facilitating a more efficient transphosphorylation that leads to enhanced biological activities for type III IFNs.

FIG. 4 shows a schematic protocol for engineering mutant receptors (IFNαR1).

FIG. 5 shows a schematic protocol for engineering mutant receptors (IL10Rβ).

FIG. 6 shows a schematic protocol for engineering IFNλ1 receptors with rotated intracellular register. Left: a schematic protocol using a lentiviral system to produce cell lines with engineered IFNλ1 receptors (e.g., HEK 293F cells that are non-responsive to IFNλ1). Right: flow cytometry data showing receptor expression levels in cells post-transduction with indicated receptors.

FIG. 7 shows that modifications in the geometry of IFN-AR1 modulate pSTAT1 responses. Left: Comparison of Emax values induced in the wild-type vs mutant IFN-λR1 expressing cells by 1 μM each of IFN-ω (arrow, dark grey), IFN-23 (black) or H11 (grey). All values were normalized to the wild-type receptor expressing cells treated with IFN-λ3 (n=9). Right: Relative quantification of pSTAT1 staining in cells expressing either the wild-type (dashed) or mutant IFN-λR1 with 2 (grey dashed line) or 3 alanine (solid) insertion by flow cytometry. Cells were treated with serial dilutions of IFN-λ3 (black circles) or H11 (grey circles) for 15 min. Curves were fit to a first-order logistic model. Error bars represent ±SEM (n=3). *p<0.05; **p<0.01; ***p<0.001; ****p<0.0001.

FIG. 8 shows the effects of downstream signaling due to modifications in the geometry of IFN-λR1. Left: Comparison of relative fluorescence instensity induced in the wild-type (dashed lines) vs mutant (solid lines) IFN-λR1 expressing Hap1 cells stimulated for 15 minutes by 1 μM each of IFN-ω (arrow, dark grey), IFN-λ3 (black) or H11 (grey). All values were normalized to the wild-type receptor expressing cells (n=3). Right: Comparison of Emax values in cells expressing either the wild-type (grey) or engineered IFN-λR1. Cells were treated with IFN-λ3, H11, or IFN-ω. Emax value of IFN-λR1 wild-type expressing cells stimulated with of IFNλ3 wild-type was assigned a value of 1. No significant differences were observed.

FIG. 9 shows an antiviral assay that demonstrates that register optimization improves antiviral responses against VSV infection. Left: Antiviral activity of IFNs in cells expressing either the wild-type (dashed line) or mutant IFN-λR1 with 2 (grey dashed line) or 3 alanine (solid line) insertion. Cells were incubated with serial dilutions of IFN-λ3 (arrow, dark grey), H11 (grey) or IFN-ω (black) for 24 h prior to VSV-GFP viral infection at 80,000 PFU/well. Fluorescence levels were recorded 18 h post-infection. Curves were fit to a first-order logistic model. Error bars represent ±SEM (n=3). Right: Treatment schematic and table summarizing the EC50 values (nM) of the antiviral assay and calculated fold-changes relative to IFN-ω treated wild-type IFN-λR1 expressing cells (assigned value 1).

FIG. 10 shows an antiproliferation assay that demonstrates that mutant IFN-λR1 receptors with a 3 alanine insertion upregulate antiproliferative activities of type III IFNs. Left: Antiproliferative activity of IFNs in cells expressing either the wild-type (dashed line) or mutant IFN-λR1 with 2 (grey dashed line) or 3 alanine (solid line) insertion. Cells were incubated with serial dilutions of IFN-λ3 (black), H11 (orange) or IFN-ω (arrow, dark grey) for 4 days. Curves were fit to a first-order logistic model. Error bars represent ±SEM (n=3). Right: Treatment schematic and table summarizing the EC50 values (nM) of the antiproliferative assay and calculated fold-changes relative to IFN-ω treated wild-type IFN-λR1 expressing cells (assigned value 1).

FIGS. 11A-11H show that induction of antiviral and antiproliferative genes is upregulated in 3 alanine inserted IFN-λR1 receptor expressing cells. PCR quantification of fold changes in induction of ISG15 (11A), MX1 (11B), SAMD9L (11C), and APOL3 (11D) cells at 6 h and ISG15 (11E), MX1 (11F), SAMD9L (11G), and APOL3 (11H) cells at 24 h post treatment with 100 nM each of IFN-λ3 (black), H11 (grey), or IFN-ω (dark grey) in wild-type or mutant cell lines. Mean changes #SEM in gene expression were determined relative to untreated cells (light grey, assigned value of 1) and normalized to 18S (n=4).

FIGS. 12A-12D show that a human transcriptome analysis over 20,000 genes reveals that differential gene expression (DEG) profile of mutant IFN-λR1 cells treated with high-affinity IFN-λ3 H11 ligand is near identical to the profiles of cells treated with IFN-ω. 12A, PCA plot showing the distribution of WT and mutant IFN-λR1 cell clusters treated with IFNω, IFNλ3, and H11 ligands for 24 h. 12B, A heatmap showing the pathways involved in PCA analysis. 12C, Venn diagrams comparing the number of upregulated genes in cells expressing either WT (left) or 3 alanine inserted mutant (right) IFN-λR1 treated with indicated IFNs. 12D, A bar plot showing the quantification of DEGs in IFN-treated cells compared to untreated controls.

FIGS. 13A-13F depict volcano plots showing decreased (dashed arrow) and increased (solid arrow) gene expression levels in cells expressing either 13A-13C, WT or 13D-13F, 3 alanine inserted mutant IFN-λR1 treated with IFNω, H11, or IFNλ3 (left to right) compared to untreated controls. DE cutoffs were set at a log 2 fold change of |1| and adjusted p value <0.01.

FIGS. 14A-14E show K-means analysis indicating six distinct enriched clusters. 14A, Heatmap showing the mean expression levels of 2,400 most variable genes across six sample sets (N=2). 14B-14E, Bar plots detailing the enrichment pathways in the curated clusters. Bar size represents gene ratios within each enriched pathway, and color represents the −Log 10 p value of enrichment. Increases in-log 10 p value are indicative of increased statistical significance.

FIGS. 15A-15G demonstrate a heatmap (15A) representation of activation levels of individual ISGs in cells expressing WT or 3 alanine inserted IFN-λR1 treated with indicated IFNs. 15B, Log 2-transformed relative expression of select antiviral ISGs including IFIT1. 15C, RSAD2, 15D, OAS1 and antiproliferative ISGS including 15E, IFI27, 15F, IFI44, and 15G, UBA7 in wild-type or mutant cell lines treated with IFN-λ3 (black), H11 (grey), or IFN-ω (dark grey). Statistical significance was determined by two-way ANOVA test. *p<0.05; **p<0.01; ***p<0.001; ****p<0.0001.

FIG. 16 shows an IPA pathway analysis which reveals that high-affinity H11 ligand induces similar subsets and fold-changes of potent antiviral ISGs as IFNω in 3 alanine inserted IFN-λR1 expressing cells. Bubble plot representation of significantly enriched antiviral mechanisms using IPA. Bubble color represents activation Z scores, and bubble size represents the −Log 10 p value of enrichment. Statistical significance was determined by an activation Z score >|1| and a −log 10 p>1.32, which correspond to a p value of 0.05. Increases in −log 10 p value are indicative of increased statistical significance.

FIG. 17 demonstrates that cells expressing high-affinity IL-10Rβ receptors induce stronger pSTAT1 responses. Left panel: Relative quantification of pSTAT1 staining in cells expressing either the wild-type (dashed line) or engineered IFN-αR1 receptors (solid line) by flow cytometry. Cells were treated with serial dilutions of IFN-ω (arrow, dark grey), IFN-λ3 (black) or H11 (grey) for 15 min. Curves were fit to a first-order logistic model. Right panel: Comparison of Emax values induced in the wild-type (grey) vs engineered IL-10Rβ (black) expressing cells by 2 μM of each indicated IFN. All values are normalized to the wild-type receptor expressing cells treated with IFN-λ3. Error bars represent ±SEM (n=3). *p<0.05; **p<0.01; ***p<0.001; ****p<0.0001.

FIGS. 18A-18E show modifications in the geometry of IFNλR1 modulate pSTAT1 responses. 18A, Schematic diagram of alanine insertion mutagenesis of the IFNλR1 transmembrane domain. 18B, α-helical wheel projections of the register rotations introduced by addition of each alanine residue are shown (top) and alanine residues (ranging from 1 to 4) were inserted after V242 (bottom; SEQ ID NO: 1). The direction of rotation is arbitrarily assigned with each residue adding a 109° rotation. 18C, Comparison of Emax values induced in the wild-type vs mutant IFNλR1 expressing cells by 1 μM each of IFNω (arrow, dark grey), IFNλ3 (black) or H11 (grey). All values were normalized to the wild-type receptor expressing cells treated with IFNλ3 (n=9). 18D, Relative quantification of pSTAT1 staining in cells expressing either the wild-type or 18E, mutant IFNλR1 with 3 alanine insertion by flow cytometry. Cells were treated with serial dilutions of IFNω (arrow, dark grey), IFNλ3 (black) or H11 (grey) for 15 min. Curves were fit to a first-order logistic model. Error bars represent ±SEM (n=3). *p<0.05; **p<0.01; ***p<0.001; ****p<0.0001.

FIGS. 19A-19F demonstrate that register optimization improves type III IFN antiviral response against VSV infection. 19A, Schematic diagram summarizing the optimization strategies and their respectively associated EC50 values (nM) of the antiviral assay and calculated fold-changes relative to IFNω treated wild-type IFNλR1 expressing cells (assigned value 1). 19B, Schematic diagram depicting the antiviral assay set up. 19C, Antiviral activity of IFNs in cells expressing either the wild-type or 19F, optimized IFNλR1. Cells were incubated with serial dilutions of IFNλ3 (black), H11 (grey) or IFNω (arrow, dark grey) for 24 h prior to VSV-GFP viral infection at 80,000 PFU/well. Fluorescence levels were recorded 18 h post-infection. Curves were fit to a first-order logistic model. Error bars represent ±SEM (n=3). 19D, PCR quantification of fold changes in induction of ISG15, 19E, MX1 at 24 h post treatment with 100 nM each of IFNλ3 (black), H11 (grey), or IFNω (arrow, dark grey) in wild-type or optimized cell lines. Mean changes±SEM in gene expression were determined relative to untreated cells (light grey, assigned value of 1) and normalized to 18S (n=4).

FIGS. 20A-20F show that optimization of IFNλR1 upregulates anti-proliferative activities of type III IFNs. 20A, Schematic diagram summarizing the optimization strategies and their respectively associated EC50 values (nM) of the anti-proliferative assay and calculated fold-changes relative to IFNω treated wild-type IFNλR1 expressing cells (assigned value 1). 20B, schematic diagram showing the experimental set-up of the assay. 20C, Anti-proliferative activity of IFNs in cells expressing either the wild-type or 20F, optimized IFNλR1. Cells were incubated with serial dilutions of IFNλ3 (black), H11 (grey) or IFNω (arrow, dark grey) for 4 days. Curves were fit to a first-order logistic model. Error bars represent ±SEM (n=3). 20D, PCR quantification of fold changes in induction of SAMD9L, 20E, APOL3 at 24 h post treatment with 100 nM each of IFNλ3 (black), H11 (grey), or IFNω (arrow, dark grey) in wild-type or optimized cell lines. Mean changes±SEM in gene expression were determined relative to untreated cells (light grey, assigned value of 1) and normalized to 18S (n=4).

FIG. 21 shows relative quantification of pSTAT1 staining in cells expressing mutant IFNλR1 with 2 alanine insertion by flow cytometry. Cells were treated with serial dilutions of IFNω (arrow, dark grey), IFNλ3 (black) or H11 (grey) for 15 min. Curves were fit to a first-order logistic model. Error bars represent ±SEM (n=3).

FIG. 22 demonstrates anti-viral activity of IFNs in cells expressing mutant IFNλR1 with 2 alanine insertion. Cells were incubated with serial dilutions of IFNλ3 (black), H11 (grey) or IFNω (arrow, dark grey) for 4 days. Curves were fit to a first-order logistic model. Error bars represent ±SEM (n=3).

FIG. 23 demonstrates anti-proliferative activity of IFNs in cells expressing mutant IFNλR1 with 2 alanine insertion. Cells were incubated with serial dilutions of IFNλ3 (black), H11 (grey) or IFNω (arrow, dark grey) for 4 days. Curves were fit to a first-order logistic model. Error bars represent ±SEM (n=3).

FIGS. 24A-24B show anti-proliferative activity of IFNs in MCF-7 cells expressing optimized (24A), or wild-type IFNλR1 receptors (24B). Cells were incubated with serial dilutions of IFNλ3 (black), H11 (grey) or IFNω (arrow, dark grey) for 4 days. Curves were fit to a first-order logistic model. Error bars represent ±SEM (n=3).

FIGS. 25A-25D show the therapeutic evaluation of a potentiated IFNλ using the engineered IFNλ receptor the in an MCF-7 tumor model. Athymic mice were inoculated with 5×10⁶ wild type or receptor-engineered IFNλR1 expressing MCF-7 human breast cancer cells subcutaneously on day 0 and treated with 100 μL PBS (intraperitoneally), n=6), 30 μg IFNλ3 (WT, IP, n=6), or 30 μg IFNλ3 (H11, IP, n=6) on days 10, 16, and 33. 25A shows the experimental schedule and 25B shows the tumor growth curve and where the average tumor volume was greatly smaller for mice treated with IFNλ3 H11 and MCF-7 tumors expressing IFNλR1 with 3 alanines than the MCF-7 cells expressing the wild-type IFNλR1 and treated with either PBS or the wild-type IFNλ3.

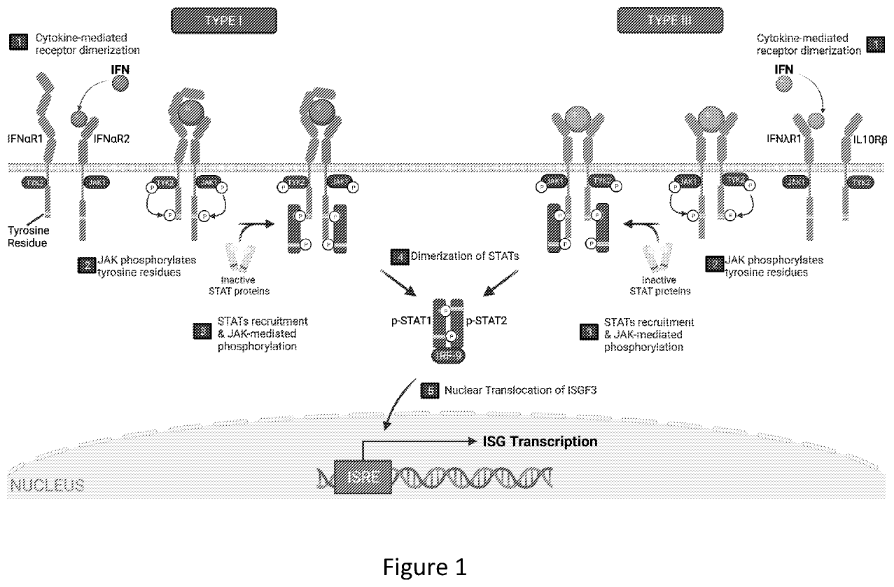

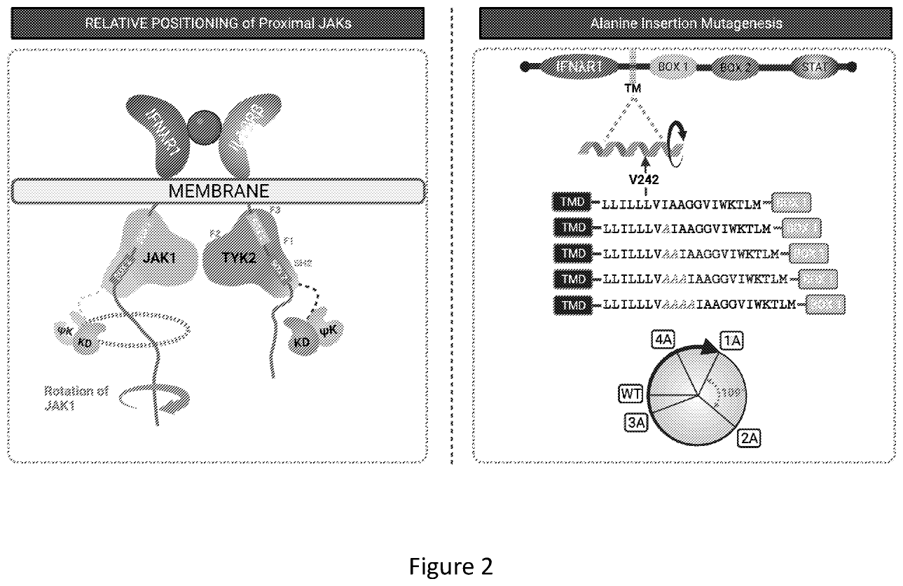

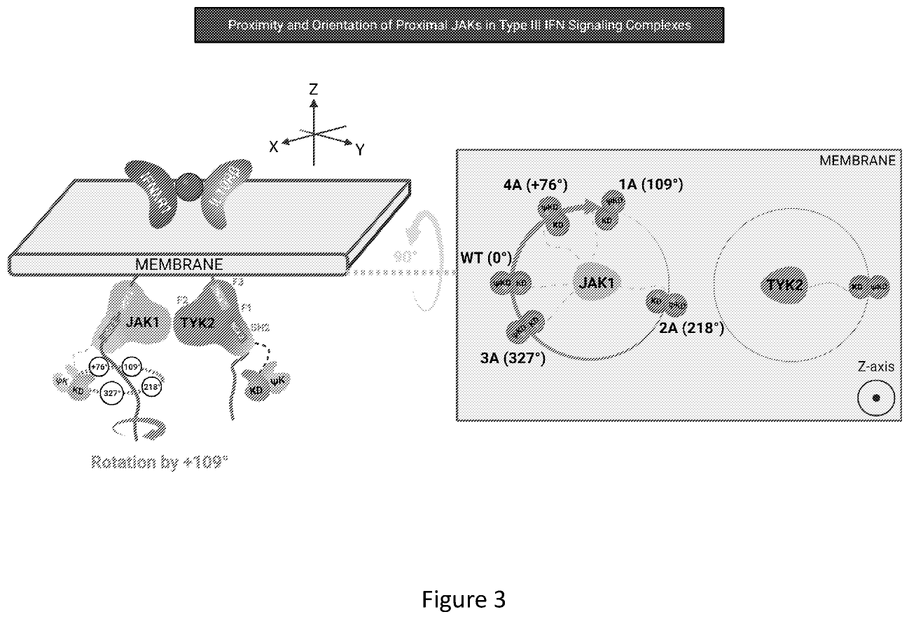

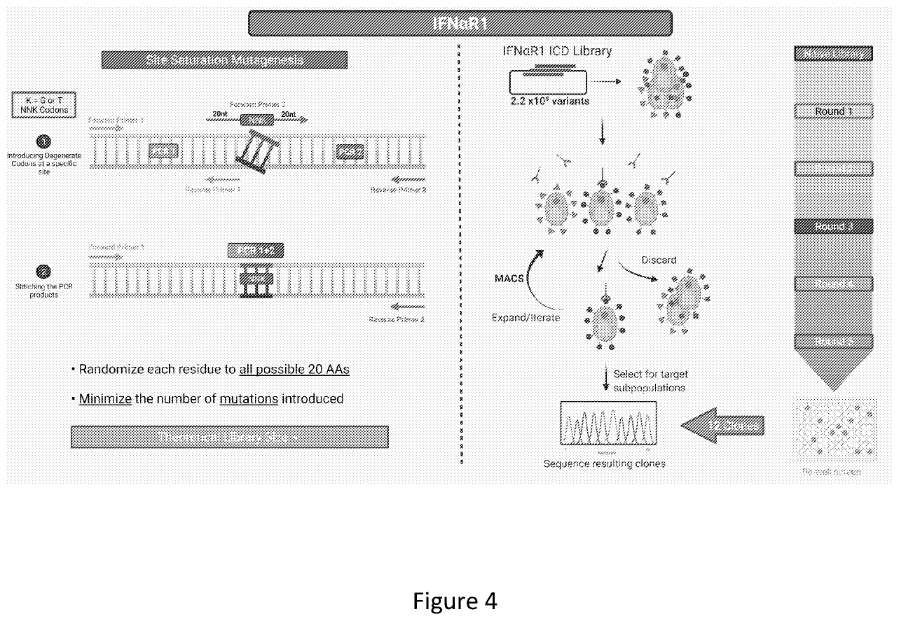

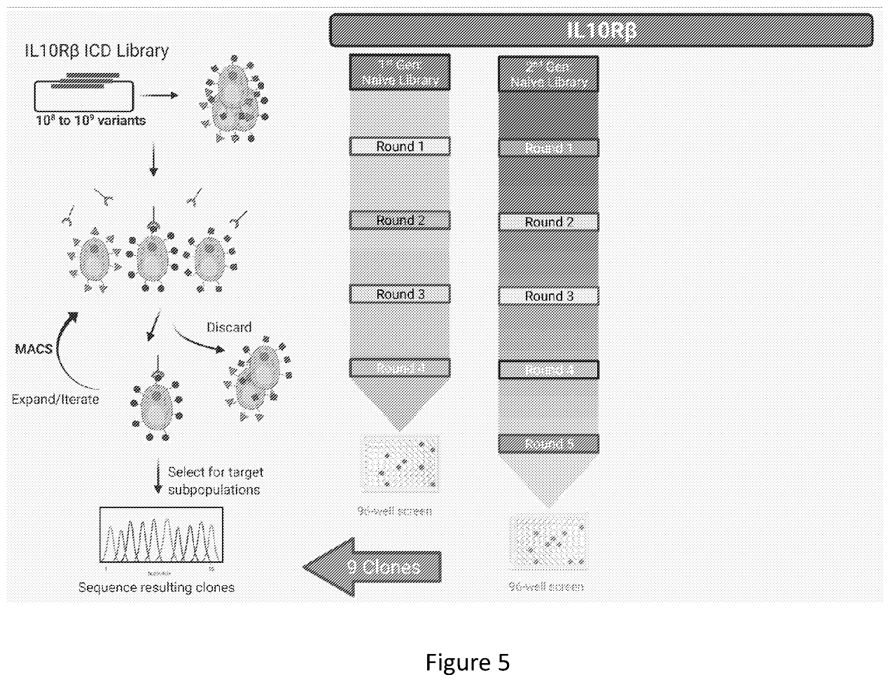

### DETAILED DESCRIPTION

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indictates otherwise.

In some embodiments, percentages disclosed herein can vary in amount by #10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to +10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Patient” refers to a warm blooded animal such as a mammal, preferably a human, which is afflicted with, or has the potential to be afflicted with one or more diseases and disorders described herein.

In view of the present disclosure, the methods and compositions described herein can be configured by the person of ordinary skill in the art to meet the desired need.

### Overview

Disclosed herein are approaches to refine cytokine-receptor interactions to tune cytokine signaling, gene expression patterns, and function to provide improved compositions and methods for treating diseases, such as cancer, viral infections, and others.

The inventor has found that affinity-matured Type III IFNs and their variants, called “super-IFN-Lambda” exhibit improved anti-viral efficacy which has been validated in a relevant in vivo mouse model. However, the “super-IFN-Lambda” remained less active than type I IFNs. This was found to be due to a difference in signaling amplitude.

Here, the three factors that limit potency of IFN lambda signaling and function have been elucidated: 1) affinity of the extracellular ligand-receptor complex (as previously shown), 2) affinity of the IL10Rβ-TYK2 interaction, and 3) the relative geometry of the JAKs in the IFNL receptor complex is not optimal.

Therefore, the present disclosure demonstrates the utility of broadly tuning all cytokine signaling and functional potency, for example, at the level of modulation of the affinity of extracellular ligand-receptor complex, (2) the affinity of receptor-kinase (e.g., IL10Rβ-TYK2) interaction, and (3) the relative geometry of kinases and receptor (e.g., JAKs and IFNL). In some embodiments, these concepts are broadly demonstrated herein as applied in the context of Type III IFN (IFNλ), but the present disclosure further contemplates similarly applying the inventive concepts described herein for any other cytokine.

For example, in order to potentiate activity of IFNλs, molecules that modulate factors 2 and 3 above or an engineered ligand that is able to modulate any combination of factors 1-3 is contemplated. From this work, such a ligand could be in the form of a IFN % that oligomerizes more than two receptors in the complex or a ligand that dimerizes IFNλR1 and IL10RB in a geometry to optimize JAK-TYK2 activity. Moreover, therapeutic agents that potentiate signaling through a cytokine receptor complex to provide a therapeutic effect are contemplated herein.

Similarly, without wishing to be bound by theory, it is believed that “tuning” cytokine receptors, for example, by changing the geometry of the receptor complex potentiates signaling through receptor complexes to provide a basis for new classes of molecules that can be engineered proteins, therapeutic molecules (e.g., small molecules, etc.), and/or other novel designed proteins. In other words, the present disclosure establishes for the first time that through “tuning” cytokine receptors (i.e., at the levels of ligand-receptor affinity, receptor-kinase affinity, and/or relative geometries of kinases and receptors), cytokine signaling can be controlled beyond the natural system (irrespective of the cytokine receptor-ligand complex in question). This tuning process can be done at the receptor level and at the ligand level by engineering new ligands that alone or in concert with one or more other therapeutic agents impose a geometric shift on a cytokine receptor complex to potentiate signaling through the receptor complex to provide a desired therapeutic effect.

In some embodiments, the present disclosure contemplates potentiating cytokine signaling, partial agonism of cytokine signaling, and inhibition of cytokine signaling by the methods and concepts disclosed herein and substantiated by the examples below.

In other embodiments, the present disclosure provides methods for screening wild-type or modified cytokines and/or candidate therapeutic agents for therapeutic effects by measuring modulation of cytokine signaling when administered to a cell harboring a wild-type cytokine receptor target. For example, a modified cytokine or candidate therapeutic agent (e.g., an agonist or antagonist of the cytokine that has been modified) can be administered to a cell harboring the cytokine receptor being targeted and changes in signaling can be measured and compared to control (i.e., wild-type cytokine+wild-type cytokine receptor) to determine the potential for a therapeutic effect of the modified cytokine or candidate therapeutic agent. Any molecules/drugs that achieve the signaling and/or functional modulation demonstrated using this approach are contemplated herein.

In other embodiments, the present disclosure provides methods for screening candidate therapeutic agents (e.g., small molecules) that modulate JAK-cytokine receptor affinities to provide a therapeutic effect. Similarly, it is envisioned that modified cytokines can be combined with such candidate therapeutic agents (e.g., small molecules) to achieve a therapeutic effect. Therefore, combinations of such therapeutic agents are contemplated herein as pharmaceutical compositions, but the agents may be provided individually.

In other embodiments, the present disclosure provides methods for screening wild-type or modified cytokines and/or candidate therapeutic agents for therapeutic effects by measuring modulation of cytokine signaling when administered to a cell harboring a genetically-modified cytokine receptor target (e.g., having a modulated ligand affinity, a modulated JAK affinity, and/or a rotated orientation that alters down-stream signaling). For example, a modified cytokine or candidate therapeutic agent (e.g., an agonist or antagonist of the cytokine that has been modified) can be administered to a cell harboring the cytokine receptor being targeted and changes in signaling can be measured and compared to control (i.e., wild-type cytokine+genetically-modified cytokine receptor) to determine the potential for a therapeutic effect of the modified cytokine or candidate therapeutic agent. Any molecules/drugs that achieve the signaling and/or functional modulation demonstrated using this approach are contemplated herein.

In other embodiments, it is contemplated that the technology described herein can be incorporated into CAR T cells to modulate CAR T cell activity. For example, modulation of CAR T cell activity can be achieved by modulation of ligand-receptor affinity, receptor-kinase affinity, and/or relative geometries of kinases and receptors. For example, affinity and/or geometry modulation, as described herein, can happen within the binding domain, spacer region, and signaling region.

In another embodiment, therapeutic agents that decrease or increase signaling through a cytokine receptor complex to provide a therapeutic effect are contemplated herein.

In some embodiments, the present disclosure is directed to an engineered IFNλ receptor.

In some embodiments, the present disclosure is directed to an engineered cytokine receptor, wherein the receptor has an altered geometry that potentiates signaling when the receptor is bound by a ligand.

In some embodiments, the present disclosure is directed to methods of treating a patient for cancer and/or a viral infection. The method can include administering to the patient a therapeutically effective amount of a therapeutic agent that potentiates signaling through a cytokine receptor complex to provide a therapeutic effect, as described herein.

In some embodiments, the cancer is melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplastic syndrome, multiple myeloma, transitional cell carcinoma, neuroblastoma, plasma cell neoplasms, Wilm's tumor, glioblastoma, retinoblastoma, or hepatocellular carcinoma.

In some embodiments, the virus causing the viral infection is HBV, HBV/HDV co-infection, Norovirus, Influenza, and/or SARS-COV2.

The present disclosure contemplates a variety of methods of administering the therapeutic agents disclosed herein, including local, oral, nasal, rectal, intravaginal, topical, subcutaneous, intradermal, intramuscular (IM), intravenous (IV), intrathecal (IT), intraperitoneal (IP), intracerebral, epidural, or intracranial administration. Local, in situ administration of these compositions is contemplated.

Other diseases and disorders are contemplated for treatment herein.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Approaches to Cytokine-Receptor Interactions to Tune Cytokine Signaling, Gene Expression Patterns, and Function

### Overview

The inventor has found that affinity-matured Type III IFNs and their variants, called “super-IFN-Lambda” exhibit improved anti-viral efficacy which has been validated in a relevant in vivo mouse model. However, the “super-IFN-Lambda” remained less active than type I IFNs. This was found to be due to a difference in signaling amplitude.

Here, the three factors that limit potency of IFN lambda signaling and function have been elucidated: 1) affinity of the extracellular ligand-receptor complex (as previously shown), 2) affinity of the IL10Rβ-TYK2 interaction, and 3) the relative geometry of the JAKs in the IFNL receptor complex is not optimal.

Therefore, the present example demonstrates the utility of broadly tuning all cytokine signaling and functional potency, as applied in the context of Type III IFN (IFNλ) by altering one or a combination of all three ways of: (1) the affinity of extracellular ligand-receptor complex, (2) the affinity of receptor-kinase (i.e., IL10Rβ-TYK2) interaction, and (3) the relative geometry of kinases and receptor (i.e., JAKs and IFNL). In order to potentiate activity of IFNλs, molecules that modulate factors 2 and 3 above or an engineered ligand that is able to modulate any combination of factors 1-3 is contemplated. From this work, such a ligand could be in the form of a IFNλ that oligomerizes more than two receptors in the complex or a ligand that dimerizes IFNλR1 and IL10RB in a geometry to optimize JAK-TYK2 activity.

### Abstract

Type I and III Interferons (IFN) constitute the host system's first line of defense against viral infections. Although the two families use distinct extracellular receptor complexes, an identical pair of Janus kinases (JAK) activate a similar set of signal transducer and activator of transcription (STATs) through a conserved pathway. Consequently, type I and III IFNs are presumed to activate a largely overlapping set of IFN-stimulated genes (ISGs) and elicit similar biological responses. Therapeutically, type III IFNs are attractive alternatives to type I IFNs due to their innate tissue specificity and lower systemic toxicity. One major limitation of type III IFNs is the significantly lower potency of their physiological activities compared to type I IFNs.

To evaluate the role of receptor geometry in IFN signaling, the inventors engineered cell lines that express wild-type and mutant IFNλ receptors (IFNλR1) with rotated intracellular register with respect to the associated JAK1. Direct evaluation of downstream signaling and biological activity of type III IFNs in cells with varied JAK-JAK geometry uncovered variant receptors that result in potentiation of type III IFN signaling and all downstream activities. In combination with the use of a high-affinity ligand to stabilize the extracellular receptor complex, it was found that the optimization of the intracellular JAK-JAK geometry enhances the type III IFN antiviral and anti-proliferative activities by 2- and 3-logs, respectively. These findings provide a molecular mechanism to explain the observed differences in potency between type I and III IFNs and provide deeper insights and new strategies for therapeutic regulation of cytokine signaling.

### Introduction

Type I and III Interferons (IFNs) are two distinct cytokine families that are crucial in arming the host system with an efficient and controlled state of immunity in response to infections (1,2). Both IFN families share important biological functions (3,4) that modulate the innate and adaptive arms of the immune system to activate gene expression programs involved in antiviral, anti-proliferative, anti-tumoral and other immunomodulatory pathways (5,6). For both families, the cytokine production is similarly induced by cellular sensing of pathogen-associated molecular patterns (PAMPs) from viral or non-viral pathogens via pattern recognition receptors (PRRs; 7,8). The upregulation of type I and III IFNs expression then initiates a chain of signaling cascades that lead to robust transcriptional induction of related IFN-stimulated genes (ISGs; 9,10). Although there is a significant overlap in the pool of ISGs induced between type I and III IFNs, multiple studies have shown that the two pathways are non-redundant, but rather have complementary functions that serve to maintain an optimal state of immune protection (11-13).

To understand and address the discrepancies in activity between the type I and type III IFNs, the previous studies on type III IFNs focused on the affinity of the ligand to its receptors composed of IFNλR1 and IL10Rβ. Through directed evolution, the inventors engineered a high-affinity variant (IFNλ3 H11) that was tested for the ability to potentiate activity (14). These studies revealed that the affinity of the receptor complex, while a contributing factor, only partly explained the lower activity of type III IFNs. More importantly, they found that the signaling amplitude (Emax) elicited by the type I IFNs is three times greater than the signal amplitude elicited by the type III IFNs. It was hypothesized that this difference in signaling amplitude likely explains the weak IFN functions such as ISG expression, antiviral and anti-proliferative/anti-cancer activities.

Gene expression studies have shown that type III IFNs induce a weaker transcriptional profile of a smaller set of ISGs compared to type I IFNs (15-17). Consequently, type III IFNs are less efficacious as antiviral or anti-tumoral agents compared to type I IFNs (18-20). Such a significant gap in potency currently presents an insurmountable hindrance in translating type III IFNs for clinical uses (21). This is reflected by the fact that no type III IFNs has been approved for clinical use whereas many type I IFNs are already clinically utilized to treat cancers, autoimmune disorders, and viral infections (22-24). However, the therapeutic gains of type I IFNs are unfortunately offset by the adverse side effects in patients (25). Given the much more favorable toxicity profile of type III IFNs largely due to the restricted expression of type III IFN receptors to epithelial and barrier cells, there is much vested research interest in understanding the key factors contributing to the lower potency of type III IFNs and developing strategies to overcome these limitations (26).

Notably, the differential signaling potency between type I and III IFNs is made more perplexing by the fact that both families share an identical intracellular Janus kinase/Signal Transducer and Activator of Transcription (JAK/STAT) signaling pathway (27,28). Unlike tyrosine kinase receptors, cytokine receptors lack intrinsic kinase domains (29). All IFNs thus utilize the JAK/STAT pathway to transmit signals to the intracellular domains to initiate signaling. For type I and III IFNs, the same pairing of JAK1 and TYK2 kinases is utilized. However, a lack of structural information regarding full-length JAK proteins in natural complexes with full-length cytokine receptors has rendered some finer aspects of the JAK/STAT pathway inaccessible. It has yet to be addressed if and how the JAK kinase domains reorient during and after ligand stimulation. Heterodimerization of receptors induced via ligand binding again prompts additional questions (30). Canonically, cell signaling is initiated when two JAKs bound to respective receptors are brought within a defined distance for transphosphorylation to occur. However, the current understanding does not make clear how the kinase domains of complex sharing JAKs are oriented with respect to each other, and if the relative orientation differs amongst distinct cytokine receptors. The inventors hypothesize that these fundamental issues concerning the intracellular geometry of JAKs can be of much significance in decoding the differences in the functional activities of type I and III IFNs as well as broader cytokine systems.

To determine if alterations to the geometric alignment of complex-sharing receptors could facilitate efficient transphosphorylation of proximal JAKs, the intracellular register of the IFNλR1-JAK1 axis was modulated and observed the effects on downstream JAK activation and subsequent signaling outputs. Alanine insertion mutagenesis was used to engineer mutant IFNλR1 receptors with precise twists in their intracellular registers that could realign the proximal JAKs within the heterodimeric signaling complex. Results indicate that the wild-type receptor contains a suboptimal alignment of transphosphorylating JAKs that is responsible for the low potency of type III IFNs relative to the type I IFNs. Functional potency of type III IFNs was found to be significantly enhanced by fine-tuning the receptor intracellular geometry. In this study, when a high-affinity ligand is utilized to stabilize the receptor complex and optimize the internal JAK geometry through receptor rotation, the type III IFN matches the type I IFN signaling in all measures of activity.

### Materials and Methods and Results

### Cell Lines and Cell Culture

Authentication of cell lines used in this study is guaranteed by the sources. SF9 cells in Sf-900™ II SFM (Gibco), Hi5 cells in Express Five™ Medium (Gibco) and HEK 293 cells in FreeStyle™ 293 Expression Medium (Gibco) were purchased from Thermo Fisher and maintained in their respective recommended media. Sf-900™ II SFM and Express Five media were supplemented with 50 μg/mL gentamicin and FreeStyle™ 293 Expression medium, with 10 U/mL of penicillin/streptomycin. Lenti-X 293T cells were cultured in DMEM+10% fetal bovine serum. All cell lines were checked for mycoplasma contamination prior to usage.

### Protein Expression and Purification

IFN-ω and IFN-λ3 were expressed and purified using baculovirus expression system, as described previously (52). Briefly, Hi5 express cells were infected with a pre-titered amount of baculovirus and cultured at 28° C. for 72 h before being harvested for proteins. The high-affinity IFN-λ3 variant, H11, was expressed similarly in HEK 293 cells. All proteins contained C-terminal hexa-histidine tags and were isolated by Ni-NTA affinity chromatography and further purified by size exclusion chromatography on a Superdex 200 column (GE Healthcare, UK), equilibrated in 10 mM HEPES (pH 7.4) and 150 mM NaCl. Proteins were stored in buffer with 10% added glycerol.

### Generation of Lentivirus Transduced Mutant Cell Lines

For the generation of lentiviral pseudoparticles, Lenti-X 293T cells were plated in 6-well plates at a density of 0.6×10⁶ cells/mL overnight. Next day, the cells were co-transfected with a plasmid encoding a cytokine receptor of interest, packaging and envelope plasmids at a fixed ratio of 0.75/0.5/0.26 μg per well, respectively. For each transfection, 4.5 μL Fugene HD transfection reagent (Promega) was combined with 1.5 μg total DNA in 100 μL of Opti-MEM (GIBCO). Cells were incubated with the transfection media for 3 days with added fresh media on day 2 before the supernatants were collected, passed through a 0.45 μm filter and stored at-80° C. in 10% FBS supplemented media. 1 mL of lentivirus containing supernatant was used to transduce 1×10⁶ target cells with fresh media being added to transduced cells every 2-3 days. On day 5, stable expression of target receptors was determined by staining against their N-terminal Flag-tag with mouse anti-Flag conjugated to Alexa 488 (Abcam).

In Vitro pSTAT1 Signaling Assay

Cells were plated overnight in a 96-well format at a density of 10,000 cells/well and treated with serial dilutions of IFN-ω, wild-type IFN-λ3 or its high-affinity variant (H11) for 15 min at 37° C. The medium was removed, and cells were detached with Trypsin (Gibco) for 5 min at 37° C. Cells were transferred to a deep-well 96-well block containing an equal volume of 4% (w/v) paraformaldehyde (PFA) solution and incubated for 15 min at room temperature. Fixed cells were then washed three times with phosphate-buffered saline containing 0.5% (w/v) BSA (PBSA), resuspended in 100% methanol for 1 h on ice. Cells were next stained with Alexa 488 conjugated pSTAT1 antibody (Cell Signaling Technology). The half-maximal response concentration (EC50) and Emax of signaling was determined by fitting the data to a sigmoidal dose-response curve (GraphPad Prism v.9).

### In Vitro Antiviral Assay

Recombinant VSV harboring a green fluorescent protein (GFP) transgene (VSV-GFP) were used. HEK 293 cells were seeded at a density of 12,500 cells/well in a 96 well format and after 45 h, the cells were treated with serial dilutions of IFN-ω, wild-type IFN-λ3 or its high-affinity variant (H11). Cell medium containing IFN treatment was removed after 24 h and VSV-GFP virus diluted in serum-free media was added to the cells at 80,000 PFU/well. At 18 h post-VSV-GFP infection, the cytopathic effects (CPE) were measured via a fluorescence plate reader.

### In Vitro Antiproliferative Assay

Cells were plated overnight in a 96-well format at a density of 10,000 cells/well. On the following day, the media was replaced with fresh media containing serial dilutions of IFN-ω, wild-type IFN-λ3 or its high-affinity variant (H11). Four days post IFN-treatment, cell density was measured using CellTiter-Glo (Promega) according to the manufacturer's protocol.

### Quantification of Gene Induction by RT-qPCR

For measuring gene induction, 600,000 cells were plated in a 6-well format overnight and treated with 100 nM each of IFN-ω, wild-type IFN-λ3 or IFN-λ3 H11 for 6 or 24 h on the following day. RNA was extracted with the Monarch Total RNA miniprep kit T2010 (NEB), 1 ug of which was converted to cDNA by a RT-PCR reaction using the High Capacity RNA-to-cDNA kit (Applied Biosystems). ISG induction relative to the untreated controls in wild-type cells was measured by qPCR assay (PowerSYBR Green PCR Master Mix, Applied Biosystems) on a QuantStudio 3 instrument (Thermo Fisher Scientific) following manufacturer's instructions. Transcription quantification was normalized to 18S internal controls. Primers were purchased from Sigma-Aldrich. See Table 1 for a complete list of primers used.

TABLE 1
List of primers used in PCR
quantification assay of ISG
gene induction

| Primer | Sequence | SEQ ID NO |
|---|---|---|
| ISG15_fwd | CGCAGATCACCCAGAAGATCG | 6 |
| ISG15_rev | TTCGTCGCATTTGTCCACCA | 7 |
| MX1_fwd | GTTTCCGAAGTGGACATCGCA | 8 |
| MX1_rev | CTGCACAGGTTGTTCTCAGC | 9 |
| APOL3_fwd | GGGACGAGTCTGGCCCTTA | 10 |
| APOL3_rev | TCAATCGGTCAATGCTGGTTG | 11 |
| SAMDOL_fwd | GAAACAGGAGCACTCAATCTCA | 12 |
| SAMD9L_rev | CAGCCTTACTGGTGATTTTCACA | 13 |
| 18S_fwd | GTAACCCGTTGAACCCCATT | 14 |
| 18S_rev | CCATCCAATCGGTAGTAGCG | 15 |

### Rna Sequencing and Transcriptome Analysis

Whole human transcriptome sequencing over 20,000 genes was performed on the Ion GeneStudio S5 Plus System using the Ion Ampliseq™ Transcriptome Gene Expression Kit (Thermo Fisher). Transcriptome libraries were barcoded, templated and sequenced using either Ion 550™ Kit-Chef and Ion 550 Chip Kit as one 16-plex library pool or Ion 540™ Kit-Chef and Ion 540 Chip Kit as one 8-plex library pool (Thermo Fisher). Two independent sequencing analyses were performed on a panel of eight samples. The RNA samples included in each panel are extracted from the following categories-untreated WT IFN-λR1 cells, untreated IFN-λR1 3A cells, IFNω treated WT IFN-λR1 cells, IFNω treated IFN-λR1 3A cells, IFN-λ3 treated WT IFN-λR1 cells, IFN-λ3 treated IFN-λR1 3A cells, H11 treated WT IFN-λR1 cells and H11 treated IFN-λR1 3A cells. Gene mapping and analysis was performed using Ion Torrent Suite™ v.5.10.0 (Thermo Fisher). Heat maps and figures showing PCA of gene expression were generated in MATLAB v.R2018b (Math Works).

### In Vivo Therapeutic Activity

MCF-7 breast cancer cells were transduced with lentivirus as previously described to induce expression of either the wild-type or IFN-λR1 3A receptor. 8-12-week old, female athymic nude mice were purchased from Jackson Laboratory and were housed at the animal facility of the University of Chicago. Animal experiments performed in this research were approved by the Institutional Animal Care and Use Committee of the University of Chicago. Wild-type MCF-7 breast cancer cell line was purchased from ATCC and cultured according to instructions. Cell lines were routinely checked for mycoplasma contamination.

Athymic nude mice were inoculated subcutaneously on the back with 5×10\hat{ }6 MCF-7 cells (or their engineered counterparts) in the presence of Matrigel (Corning). 10 days after tumor inoculation, mice received either wild-type IFNλ3 or mutated IFNλ3 (H11) intraperitoneally (30 μg/dose) in PBS in 100 μL. Negative control mice received an equal volume of PBS. Cytokine treatment was repeated on days 16 and 22 for a total of 3 treatments. Tumors were measured 2-3 times per week with digital calipers and volume was determined according to the formula: (width)×(height)×(thickness)×(pi/6). Mice were euthanized when the tumor volume was above 600 mm3 or in accordance with humane end-point criteria.

For therapeutic studies, engineered interferons in MCF-7 tumor models were used. Athymic mice were inoculated with 5×10⁶ wild type or receptor-engineered MCF-7 human breast cancer cells subcutaneously on day 0 and treated with 100 μL PBS (intraperitoneal (IP), n=6), 30 μg IFNλ3 (WT, IP, n=6), or 30 μg IFNλ3 (H11, IP, n=6) on days 10, 16, and 22.

### Statistical Analyses

Results were presented as means±standard deviation (STD). The statistical significance of differences between the groups was determined by two-way ANOVA analysis with subsequent correction for multiple comparisons using Tukey test. All statistical analyses were performed using GraphPad 9.0.2. Differences were considered statistically significant at ****p<0.0001, ***p<0.001, **p<0.01 and *p<0.05. The statistical analysis of experiments with technical replicates is detailed in figures' legends.

### Results

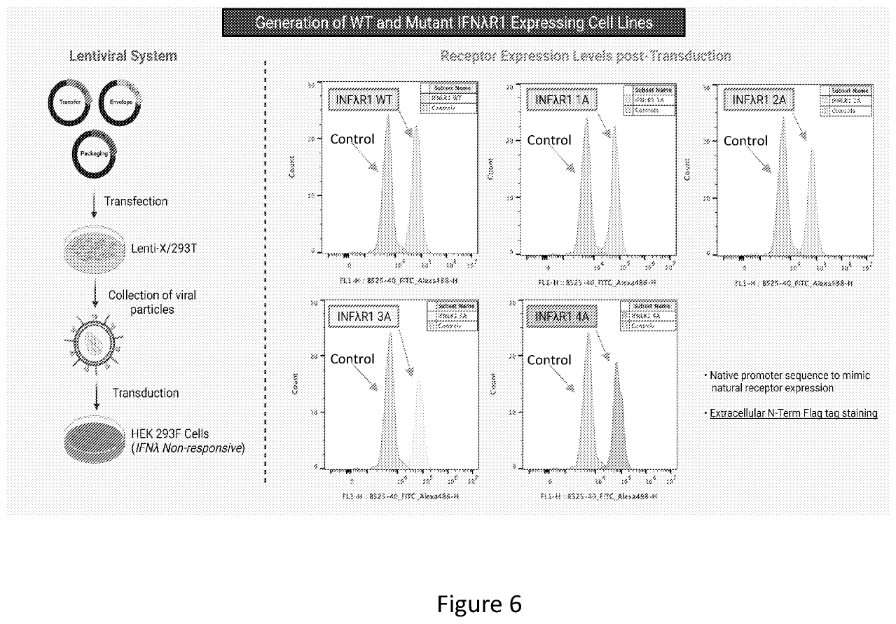

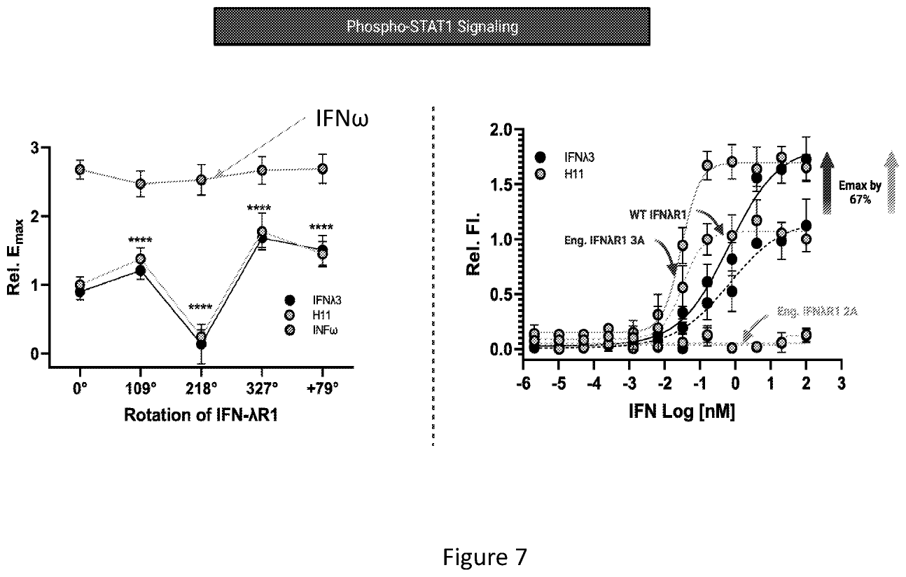

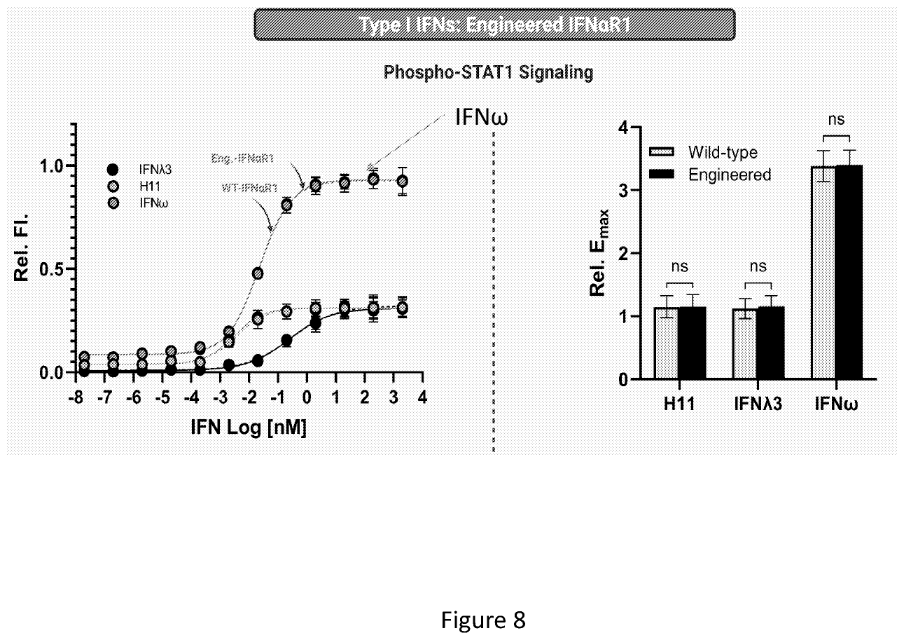

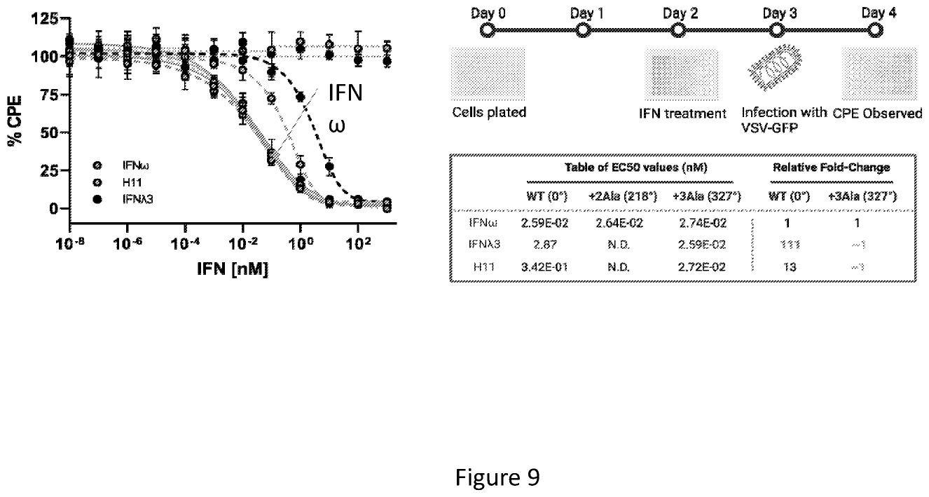

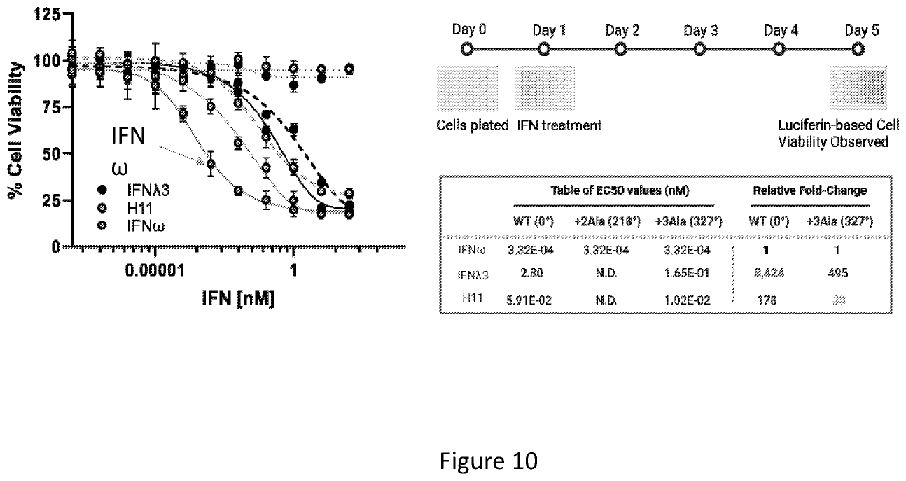

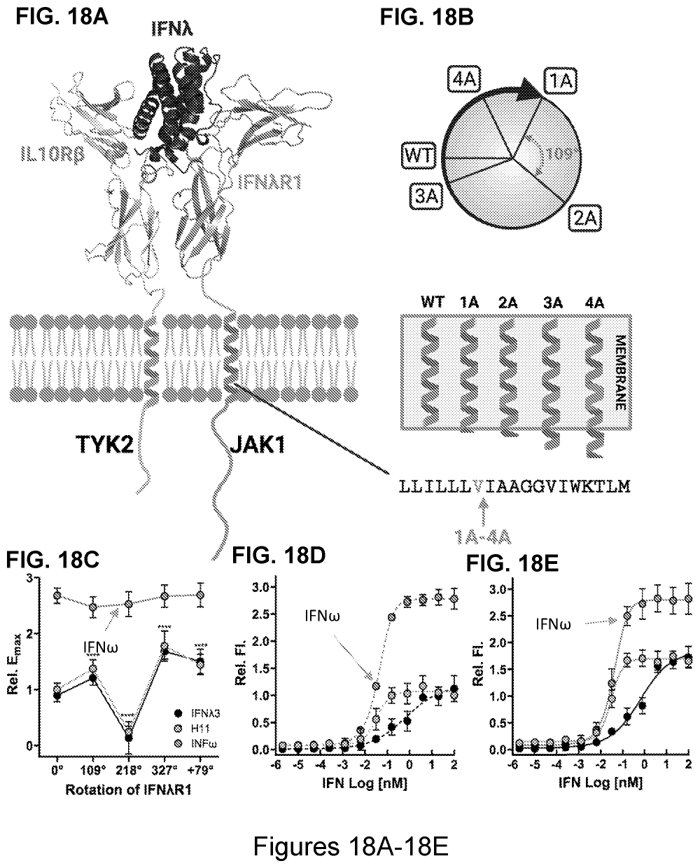

To interrogate how the geometry of proximal JAKs within a signaling complex affects type III IFN signaling, the transmembrane domain of IFNλR1 was subjected to alanine-insertion mutagenesis—an approach which has been previously utilized to alter the intracellular register of a cytokine receptor (FIG. 18A; 31, 32). Mutant IFNλR1 receptors were engineered with 1-4 alanine residues inserted after V242 within the alpha-helical transmembrane domain, with each added residue rotating the intracellular register by 109-degrees (FIG. 18B). These mutated receptors, referred to herein as λR1-1A, λR1-2A, λR1-3A, λR1-4A, were expressed stably in human embryonic kidney (HEK) 293 cells which are normally non-responsive to type III IFNs due to their very low expression levels of IFNλR1 but become responsive after being transduced to express exogenous IFNλR1 (4, 33). N-terminal Flag tags were incorporated to enable accurate quantification of receptor expression levels, which were then used to normalize functional data (FIG. 6 (right panel)).

First, the direct downstream target of IFN signaling, phospho-STAT1 (pSTAT1) levels, was measured in transduced HEK 293 cells treated with IFNω, wild-type IFNλ3 or engineered high-affinity IFNλ3 variant or H11 that was previously reported (34). As predicted, the Emax values (maximum signaling potency) of pSTAT1 induced by IFNω were largely unaffected in all cell lines (FIG. 18C). In contrast, cells treated with IFNλs, differed in their maximal signaling amplitudes. IFNλR1 receptors with 2-alanines inserted in the transmembrane domain resulted in a near total loss of pSTAT1 signaling whereas cells with 3-alanines inserted the Emax value by ˜65% compared to wild-type receptor expressing cells (FIG. 18C). Insertion of either 1 or 4-alanines also significantly improved signaling, with λR1-4A cell line slightly outperforming the former. Similar trends in the signaling magnitude were observed regardless of the use of the wild-type or high-affinity IFNλ3 ligands.

In accordance with existing literature, type III IFNs trailed significantly behind type I IFNs in terms of pSTAT1 signaling potency. The experimental Emax of IFNω was ˜2.67 fold over that of wild-type IFNλ3 in wild-type IFNλR1 expressing cells (34). Notably, the fold difference is reduced to ˜1.5 (44% reduction) by having IFNλ3 signal through the mutant IFNλR1 with three added alanine residues. It should be noted that while the signaling maxima for both IFNλ3 and H11 ligands were markedly increased by the change in the receptor orientation, EC50 values remained largely unperturbed (FIGS. 18D and 18E). Together with the inventors' previous findings, these results indicate that the geometry of the intracellular receptor-JAK complex determines the strength of the signaling while the stability of the extracellular receptor-ligand complex, the cellular sensitivity (34).

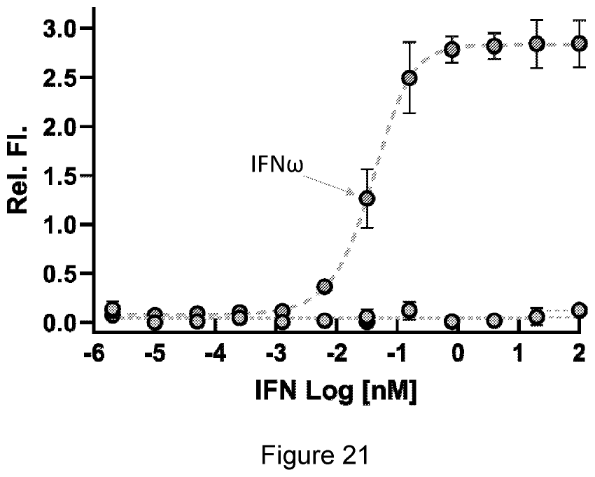

Based on the results, it can be argued that the relative positioning of intracellular JAKs in native heterodimeric complexes of type III IFNs is not optimized toward efficient downstream signaling. Here, results have shown that the intracellular register of IFNλR1 can effectively be rotated by introducing helical twists in the transmembrane domain of the receptor. The consequences of the resultant rotations are particularly evident in cells expressing IFNλR1-2A (FIG. 21). In this cell line, pSTAT1 signaling is virtually lost due to a near 180-degree flip in the orientation of JAK1 with respect to TYK2, which likely poses a physical impossibility for the JAKs to transphosphorylate. On the contrary, when the register of IFNλR1 ICD is offset by a predicted 327-degree from its native position, a significant gain in the signaling amplitude was observed. Taken collectively, it can be concluded that for optimal type III IFN signaling, the complex-sharing JAKs must not only be within a defined distance but also that the proximity must be complemented with the proper register through which the cytokine signaling can be tuned.

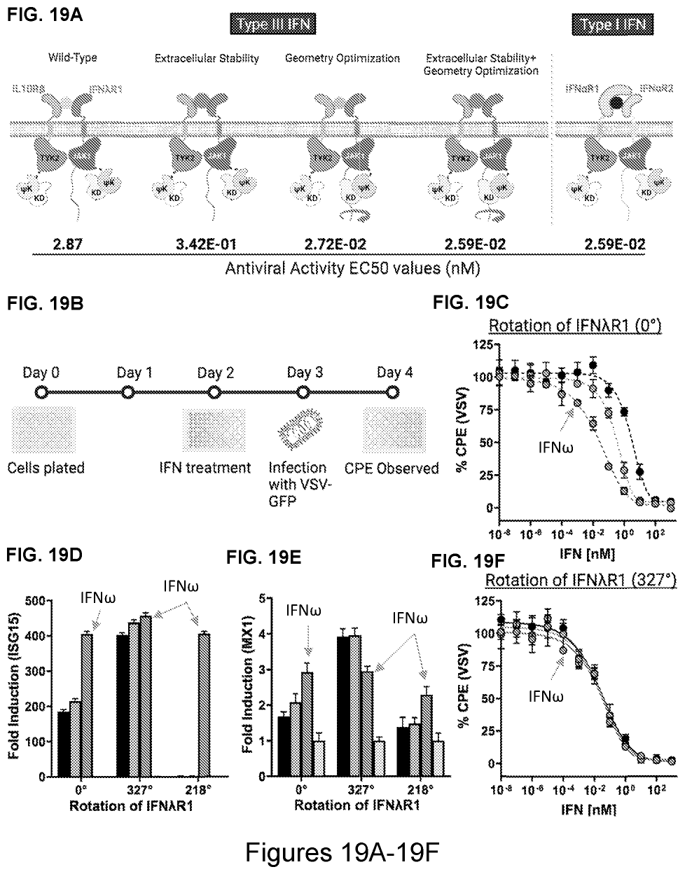

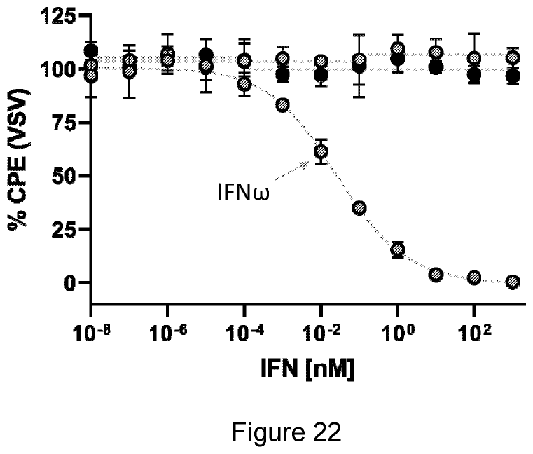

Antiviral signaling assays were used to determine if the antiviral potency of type III IFNs are improved by tuning the signaling amplitude through the use of geometry-optimized IFNλR1 receptors. The wild-type and optimized IFNλR1 expressing cell lines were infected with a recombinant vesicular stomatitis virus linked to a green fluorescent protein construct (VSV-GFP) (FIG. 19B). Consistent with the pSTAT1 signaling profiles, type III IFNs showed complete loss of antiviral activity in the IFNλR1-2A cells (FIG. 22) whereas IFNω induced similar antiviral responses across all cell lines (FIGS. 19C and 19F). Interestingly, the wild-type IFNλ3 and its high-affinity variant, H11, which were 84 and 12-fold lower in activity (EC50) than IFNω respectively in wild-type IFNλR1 expressing cells, effectively matched their antiviral activities to IFNω in IFNλR1-3A expressing cells (FIG. 19A).

A previous study conducted in HCV-infected Huh7.5 cells reported that the antiviral activities of type III IFNs can be improved 12-fold by a 150-fold improvement in the stability of the extracellular heterodimeric receptor complex through the engineered high-affinity ligand, H11 (34). However, despite the significant gain in antiviral activity, the EC50 for H11 remained 10-fold greater than IFNω (34). Here, it can be observed that the antiviral activity of type III IFNs is highly responsive to the increased pSTAT1 signaling potency modulated by the change in the intracellular IFNλR1 register. Notably, results indicate that optimization of intracellular receptor-JAK geometry can potentiate the antiviral activities of type III IFNs, regardless of their receptor affinities, to similar extents achieved with type I IFNs.

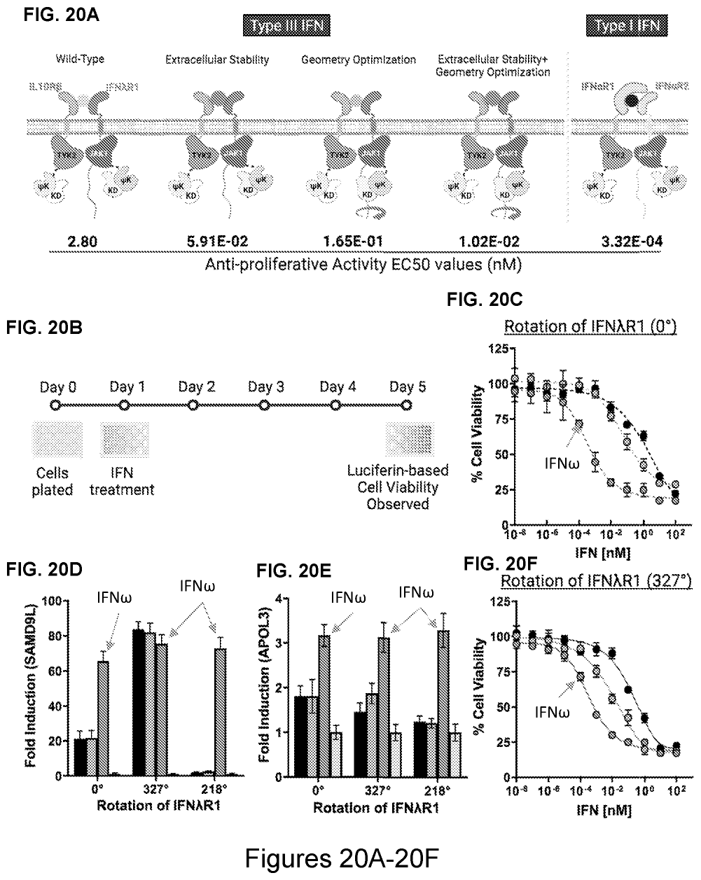

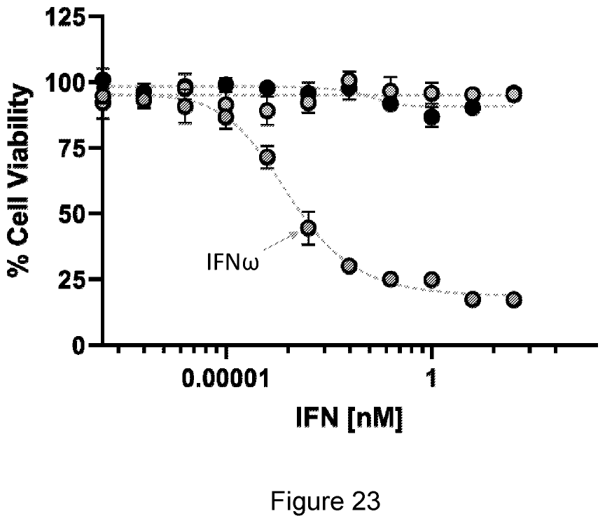

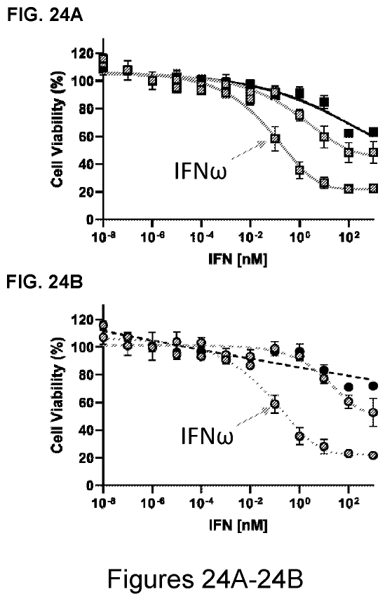

In addition to their most prominent role as antivirals, type I IFNs are also known for their anti-tumor properties (35). Here, it was observed that the anti-proliferative activities were most efficiently induced by IFNω across all cell lines (FIGS. 20C and 20F). The activity of IFNω is approximately ˜8,500-fold over that of wild-type IFNλ3 signaling through wild-type IFNλR1. Analogous to pSTAT1 signaling, IFNλR1-2A expressing cells displayed negligible antiproliferative activity when treated with IFNλs (FIG. 23). Remarkably, the near 4-log difference in activities between the type I and III IFN was reduced to just ˜30-fold in optimized IFNλR1 expressing cells stimulated with high-affinity H11 ligand, which represents a >280-fold improvement in activity (FIGS. 20A and 20F).

It has previously been shown that antiproliferative activity of type III IFNs can be modulated via cell surface receptor density and the stability of the extracellular complex (33, 35-37). Here, results indicate that the geometry of the intracellular signaling components also contributes to the antiproliferative response. Notably, an analysis of the fold-changes in activity induced in different cell lines by two IFNλ ligands suggests a possible synergy between the affinity of ligand and the register of receptor-JAK complex in modulating antiproliferative activities. When evaluated on the basis of receptor usage, the fold-increase in activity in response to the change in ligand affinity was significantly lower in the wild-type cells than in optimized IFNλR1 expressing cells. Similarly, when evaluated on the basis of ligand usage, the fold-increase in response to the change in receptor geometry was significantly lower for IFNλ3 than its high-affinity counterpart, H11. Maximal antiproliferative effects were achieved only when the stimulation with high-affinity ligand was accompanied by cell signaling through geometry-optimized mutant IFNλR1 receptor.

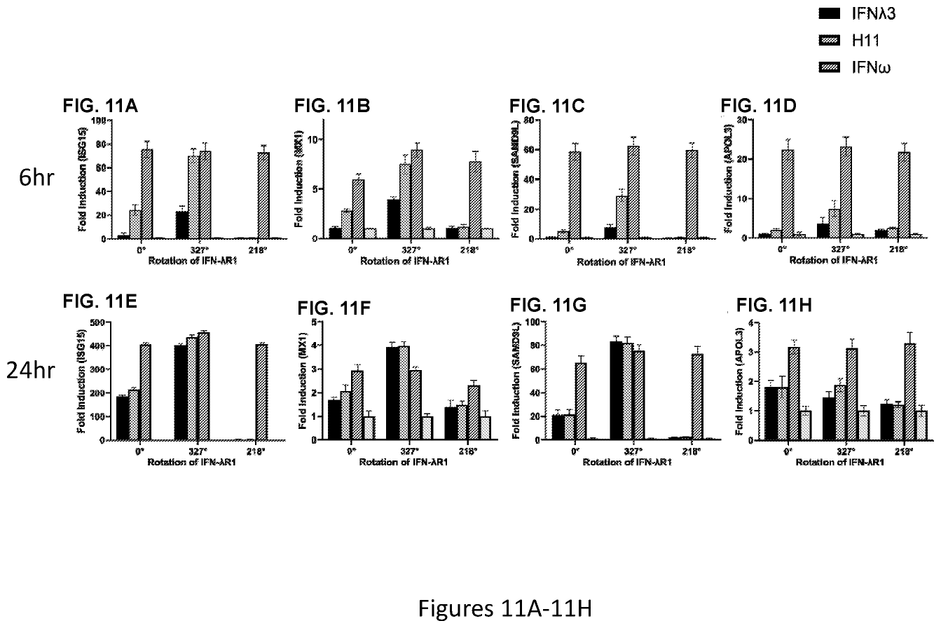

The transcriptional levels of a representative antiviral and anti-proliferative gene set were then quantified. At 6-hr post treatment with IFNs, the induction levels of antiviral genes (ISG15 and MX1), were significantly increased (3 to 8-fold) in optimized IFNλR1 expressing cells compared to the wild type (FIGS. 19D and 19E). Most notably, the gene induction levels by the high-affinity H11 effectively matched those of type I IFN, IFNω, in optimized IFNλR1 cells. Similarly, for anti-proliferative genes (APOL3 and SAMD9L), large fold-increases (4 to 6-fold) in gene induction levels by type III IFNs in the optimized IFNλR1 cells (FIGS. 20D and 20E) were observed. However, unlike the antiviral genes, the increased gene induction levels by type III IFNs, although greatly elevated, were only a fraction of those exhibited by IFNω. Notably, at 24-hr post IFN treatment, type III IFNs, regardless of their receptor affinity, induced all genes except APOL3 to near equivalent levels of type I IFN (FIGS. 11E-11H). The gene induction study again highlights the importance of receptor geometry in downstream signaling outputs by IFNs. It should be noted that the two antiviral genes screened in this assay were more sensitive to the optimized register of the receptor than the anti-proliferative genes. This is consistent with the prior functional assays evaluating the antiviral and anti-proliferative activities of IFNs in vitro.

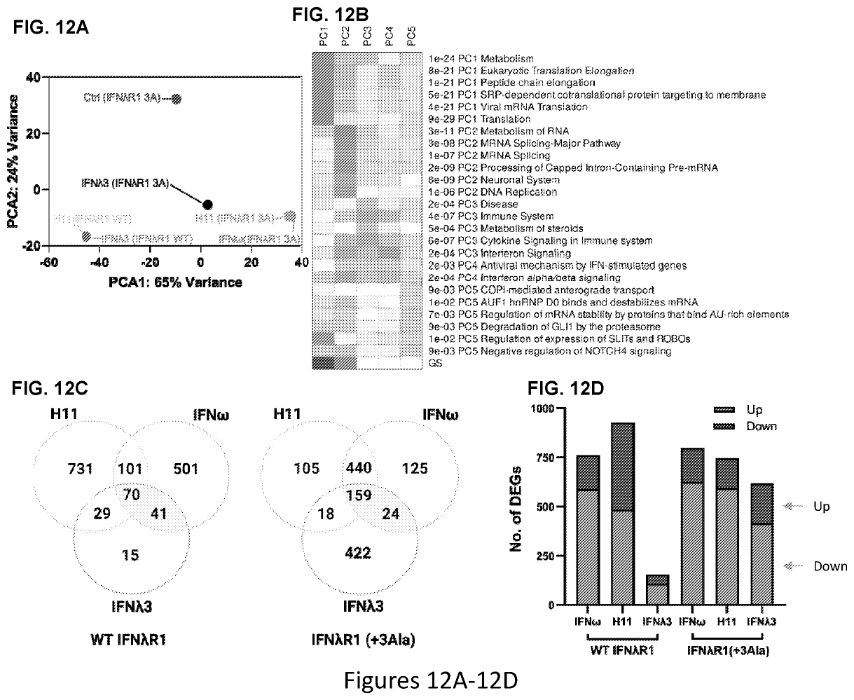

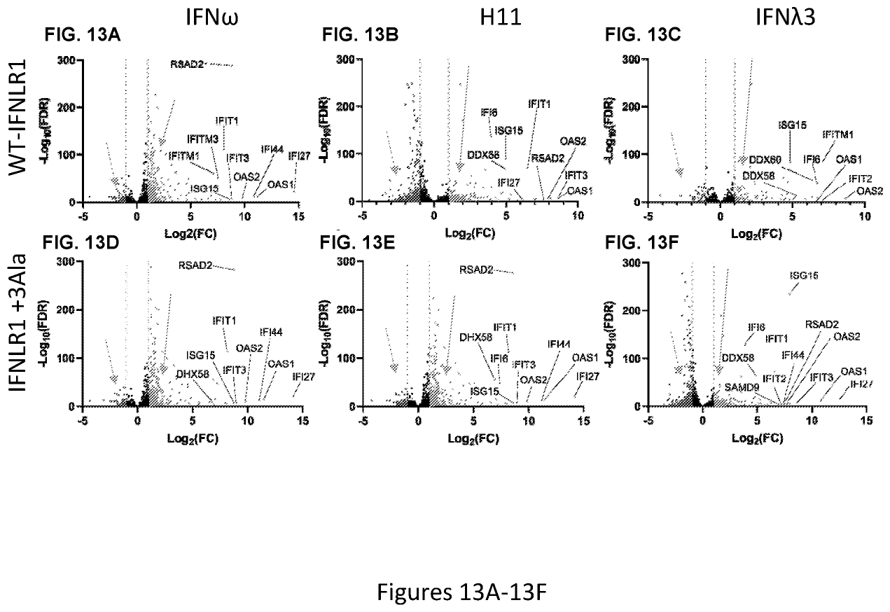

Next, next generation RNA sequencing was carried out to further evaluate the differences in the transcriptional responses to type I and III IFNs in wild-type vs optimized IFNλR1 expressing cells. Whole-genome transcriptional profiling of IFN treated cells after 24 hr displayed strong correlation with the expression patterns of select antiviral and anti-proliferative gene sets obtained by qPCR. Principal component analysis (PCA) revealed four clusters-1) untreated wild-type and optimized IFNλR1 cells, 2) IFNλ3 and H11 treated wild-type IFNλR1 cells, 3) IFNλ3 treated optimized IFNλR1 cells, and 4) IFNω treated wild-type and optimized IFNλR1 cells and H11 treated mutant IFNλR1 cells (FIGS. 12A and 12B). Compared to wild-type cells, optimized IFNλR1 cells displayed a significant increase in the abundance of overlapping differentially expressed genes (DEG) between type I and III IFNs treatments (FIG. 12C). Overall, there was a significant increase in the number of upregulated genes in response to type III IFNs in optimized IFNλR1 cells than in wild-type cells (FIG. 12D). Specifically, the number and fold-change of core antiviral ISGs were markedly improved for both IFNλ3 and H11 simulation in cells expressing optimized IFNλR1 receptors than the wild-type counterparts (FIGS. 13A-13F).

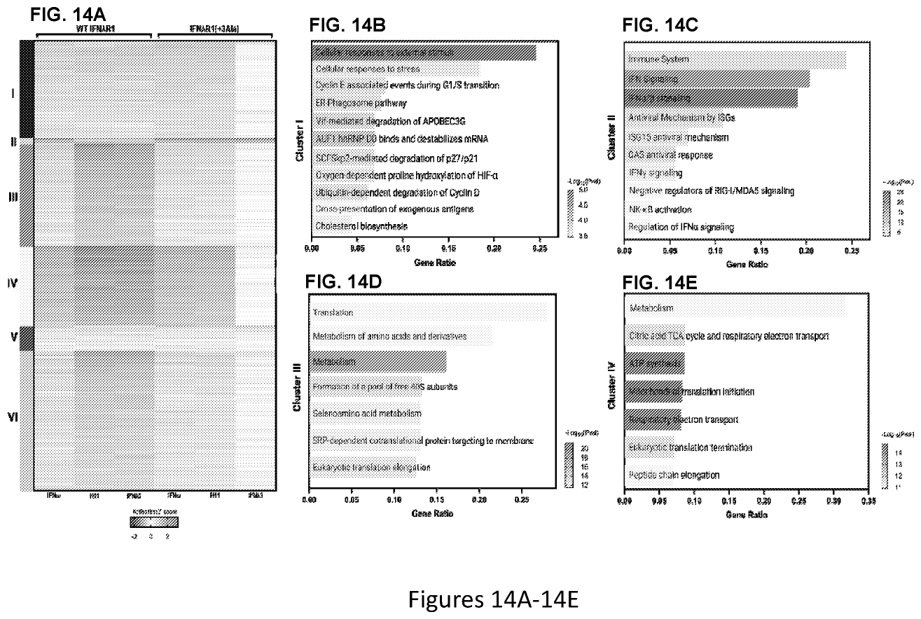

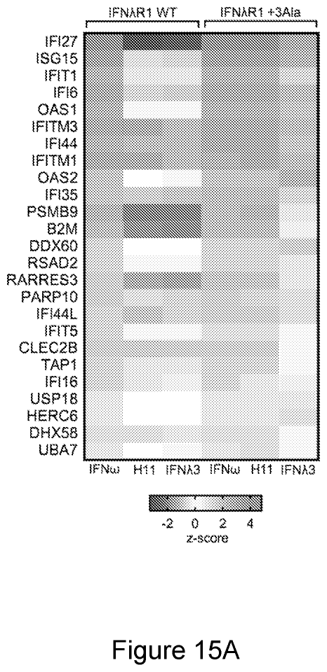

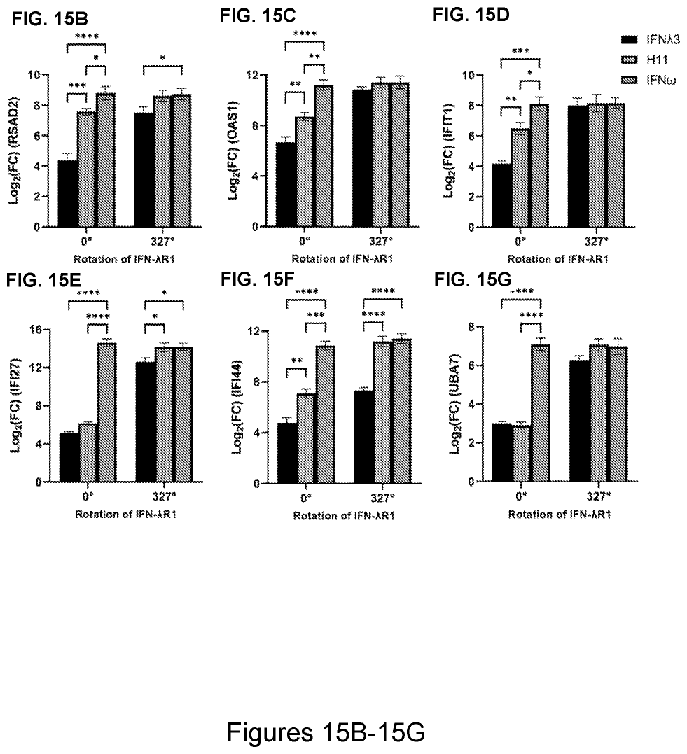

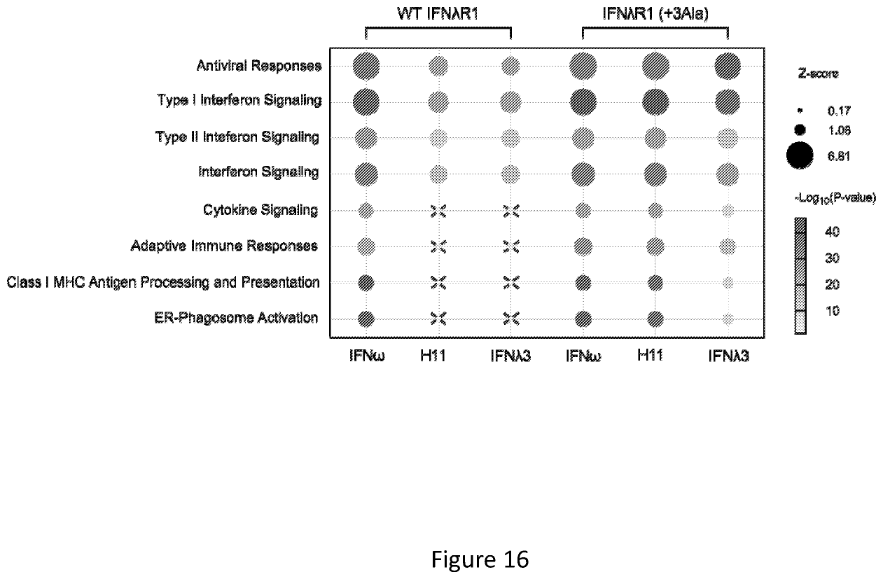

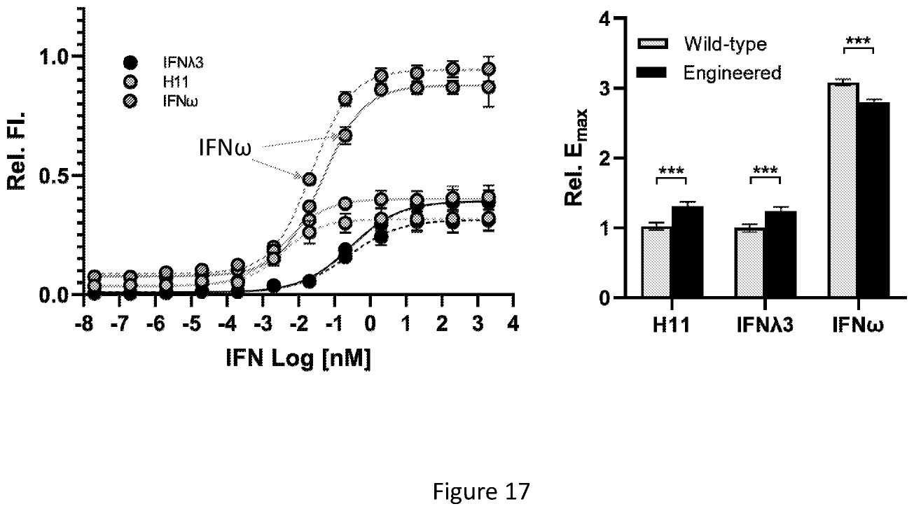

K-means clustering analysis of 2,400 most variable genes indicated six distinct enriched pathways (FIG. 14A). In both cell lines, type I IFN treatment led to the activation of genes involved in pathogen sensing and antigen processing/presentation (cluster I), innate and adaptive immune responses to viral infections (cluster II), and tissue repair and barrier functions (cluster III and IV, respectively) (FIGS. 14B-14E). On the contrary, significant differences were observed for type III IFNs in the transcriptional profiles between wild-type and optimized IFNλR1 expressing cells. As anticipated, type III IFNs induced much weaker transcriptional programs of antiviral and barrier function-associated genes than type I IFN in wild-type cells. In optimized IFNλR1 expressing cells however, stimulation with H11 led to similar activation levels of gene subsets as type I IFN across all four clusters whereas stimulation with wild-type IFNλ3 showed a marked elevation in transcription levels compared to wild-type cells, although comparatively weaker than H11 or IFNω. Similarly, the activation states of core antiviral and anti-proliferative genes displayed clear enrichment patterns for IFNω and type III IFNs signaling through rotated IFNλR1 receptors (FIG. 15A). Analysis of log 2-transformed fold changes in individual select ISGs further indicated largely equivalent gene expression profiles among different IFN treatments in optimized IFNλR1 expressing cell lines (FIGS. 15B-15G). Through Ingenuity Pathway Analysis (IPA), the activation state of individual pathways involved in maintaining a state of immunity against pathogenic stimuli (FIG. 16) was further quantified. Consistent with previous analyses, an overlap in the enrichment of genes central to IFN-mediated antiviral responses between type I IFN treated cells and H11 treated optimized IFNλR1 expressing cells were observed.

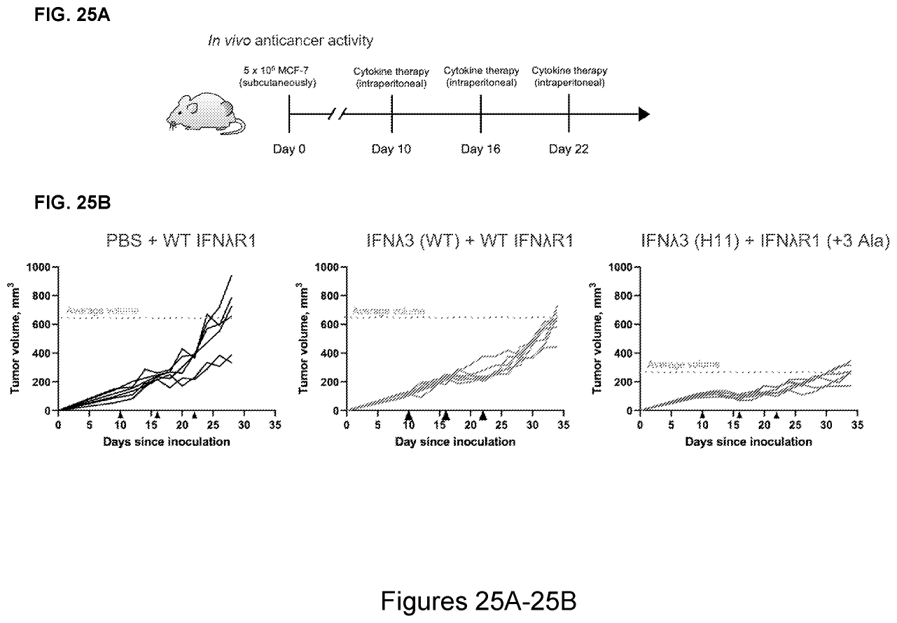

Treatment of the mice bearing wild-type MCF-7 breast cancer with wild-type IFNλ3 showed only a slight inhibition of tumor growth when compared to the saline treatment (FIG. 25B, left and middle panels). However, when mice bearing receptor-engineered MCF-7 were treated with the mutated IFNλ3 (H11), marked inhibition of tumor growth was observed (FIG. 25B, right panel). This result demonstrates that IFNλ3 (H11) mutein (mutant protein) has a higher in vivo antitumor activity on cells with engineered IFNλ receptor than the wild-type receptor-ligand pair. It is important to note that the observed antitumor efficacy is a result of direct inhibition of the tumor cells, since the human IFNλ is not expected to bind to and activate mouse host cells.

### Discussion

The intrinsic discrepancy between type I and III IFN signaling strength is of particular interest because the intracellular signaling machinery employed by the two systems is nearly identical. Unlike receptor tyrosine kinases (RTKs), type I and III IFN receptors lack intrinsic tyrosine kinase domains in the cytoplasmic regions of their polypeptide chains and signal through the JAK/STAT pathway to propagate signals to the cytoplasmic components of the cascade (38). There are four JAK kinases—JAK1, JAK2, JAK3 and TYK2—and seven STAT proteins—STAT1, STAT2, STAT3, STAT4, STAT5a, STAT5b, STAT6 (39, 40). In the canonical model, cytoplasmic tails of cytokine receptors are constitutively associated with a specific member of JAK protein via membrane-proximal binding sites, forming a complex that is functionally equivalent to RTKs (41-43). The ligand binding event oligomerizes the receptors, bringing the receptor-JAK complexes into close proximity and allowing the JAKs to transphosphorylate each other. Activated JAKs in turn phosphorylate specific tyrosine/serine residues on the cytoplasmic regions of the associated cytokine receptor, creating docking sites for the SH2 domains of STAT proteins (30, 44). Specific STATs are then phosphorylated and released to allow formation of homo- or heterodimeric STAT complexes (45, 46). These complexes then subsequently translocate to the nucleus and bind to target sequences in the genome to initiate gene transcription (47). Canonically, type I and III IFN receptors both utilize the JAK1/TYK2 kinases to form the STAT1/STAT2/IRF-9 (Interferon Regulatory Factor-9) signaling complex, known collectively as ISGF3 (Interferon-Stimulated Gene Factor 3) complex, which is an integral transcriptional regulator of core ISG genes (28).

While the stability of the extracellular ligand-receptor complexes is a contributing factor to the downstream signaling outputs, it has been shown that engineered IFNλ ligands with higher receptor affinities exhibit limited effects on bridging the potency gap between type I and III IFNs (34). In a previous study, a high affinity variant of IFNλ3 (termed H11) was engineered to increase the overall stability of the IFNλ ternary complex by 150-fold compared to the wild-type. Despite improvements in antiviral and anti-proliferative activities, the overall signaling potency and functional responses of H11 were significantly lower than those afforded by type I IFNs (34). Although receptor abundance also seems to be a limiting factor for certain IFN activities, there may be other characteristics inherent to receptor/JAK interactions that can further shed light on the differential functional capabilities of type I and III IFNs. The inventors hypothesized that there exists an optimal geometrical alignment for the juxtaposed JAKs within the same heterodimeric IFN signaling complex that favors efficient transphosphorylation. The inventors sought to address the question of whether type III IFN signaling can be made more potent by fine-tuning the geometry of their intracellular signaling complex. Hence, using alanine insertion mutagenesis approach, mutant IFNλR1 receptors were engineered with precise twists in their intracellular registers.

A series of functional assays were conducted by probing the pSTAT1 signaling, antiviral, anti-proliferative activities and ISG gene induction levels by stimulating wild-type and mutant IFNλR1 receptor-expressing cell lines with type I IFN (IFNω) and type III IFNs-wild-type IFNλ3 and high-affinity H11 ligands. The cytopathogenicity assay in cells infected with recombinant VSV-GFP virus shows that the antiviral efficacy of type I IFNs was effectively matched by type III IFNs signaling through optimized IFNλR1 receptors. This represents a 111-fold improvement in EC50 values of type III IFNs to native receptor-expressing cells. Similarly, the anti-proliferative activities of type III IFNs show significant improvements in response to the change in the receptor register. In cells expressing optimized IFNλR1 receptors, the EC50 of high-affinity IFNλ3, H11, was only 30-fold lower than that of IFNω, which is a drastic reduction of the near 4-log activity gap between IFNω and IFNλ3 in wild-type receptor-expressing cells.

Consistent with the increased pSTAT1 Emax levels and improved functional activities, the IFN-inducible gene expression is also improved in optimized IFNλR1 expressing cells. At 6 h post-stimulation with IFNs, the two antiviral genes (MX1 and ISG15) showed the most drastic differences between the wild-type and optimized receptor-expressing cell lines. Most notably, the high-affinity IFNλ3 H11 ligand was able to match the antiviral gene induction levels exhibited by IFNω. Though significantly improved by receptor reorientation, the anti-proliferative gene expression levels (SAMD9L and APOL3) of IFNλs, however, remained lower than those of type I IFNs. Interestingly, the transcriptional profiles obtained after 24 h post-stimulation showed that IFNλs, regardless of their receptor affinities, induced expression of the antiviral (MX1 and ISG15) genes and SAMD9L, an anti-proliferative related gene, to equal extents as IFNω. Whole-genome transcriptional analysis also showed that genes associated with mounting innate and adaptive immune responses against viral infections as well as maintaining tissue barrier functions were markedly upregulated in cells expressing optimized IFNλR1 receptors when treated with type III IFNs. The extent of overall gene activation by the high-affinity ligand, H11, was nearly identical to that of IFNω whereas that by wild-type IFNλ3 was weaker. However, when the analysis is limited to core antiviral ISG subsets, the wild-type IFNλ3 was able to achieve similar expression levels as H11 by the optimization of JAK-JAK geometry, which is consistent with the antiviral assay in which both IFNλ3 and H11 displayed similar EC50 values toward VSV-GFP infection. The results indicate that while geometry optimization can significantly expand the number and fold-changes of ISGs activated by type III IFNs, the optimization must be accompanied by an improved extracellular complex stability through use of high-affinity ligands in order to match the transcriptional profile achieved by type I IFNs.

In the present study, it was proposed that in the context of type III IFN signaling complexes, the rotational repositioning of the receptor bound JAK1 afforded by the insertion of 1, 3 or 4 alanine residues in the transmembrane region of IFNλR1 reorients the kinase domain of JAK1 relative to that of TYK2 bound to IL10Rβ in such a way that the activation loops from both kinase domains are brought into either a more ideal orientation and or a closer proximity. In the absence of high-resolution structures of the type I and type III IFN receptor complexes that include the intracellular JAKs, the absolute rationale as to the extent with which the increased proximity and/or the optimized orientation of JAKs influence the strength of cytokine signaling remains difficult to be determined definitively. Ultimately, the results indicate that the receptor-JAK interface adds another layer of complexity in understanding the functional differences between type I and III IFNs.

Severe acute respiratory syndrome coronavirus 2 (SARS-COV-2) global outbreak has so far claimed over 5 million people worldwide (48). The pandemic has precipitated tremendous research efforts to develop vaccines and antiviral therapeutics to curb risk of transmission, disease severity and mortality rates. Given the successful mainstream applications of type I IFNs in the treatment of chronic hepatitis B and C viruses (HBV, HCV), SARS-COV-2 outbreak has instigated renewed interest in both type I and III IFNs (22-24, 49). Thus far, a landmark study of IFNβ1a alone or in tandem with remdesivir (NCT04492475) conducted by National Institute of Allergy and Infectious Diseases (NIAID) associated worse clinical outcomes due to severe adverse events with IFNβ1a treatment (50). On the contrary, although adverse effects are notably lower with pegylated IFNλ1, phase 2 clinical trial data have so far been mixed largely due to the low therapeutic efficacy of IFNλs (21, 25). In addition to their most prominent role as antivirals, type I IFNs are also used as anti-tumor agents (35). Both the recombinant and pegylated forms of certain type I IFNs, IFNα subtypes in particular, have been in clinics for some cancers such as melanoma, hairy-cell leukemia and Kaposi's sarcoma (3). However, due to the near ubiquitous expression of type I IFN receptors in tissues, the systemic administration of type I IFNs inevitably leads to off-target side effects. Given the overlapping gene expression profile between type I and III IFNs, type III IFNs with their limited receptor distribution and tissue abundance are increasingly regarded as more specific and less toxic alternatives to type I IFNs (51). In regard to both antiviral and anticancer applications, it is evident that strategies to enhance the potency of IFNλs may have important clinical and public health implications in current and emerging epidemics as well as in the ongoing effort toward cancer therapies.

Although the present work is focused on type I and III IFNs, the engineering tools, workflow, and resultant insights into the intracellular signaling machinery can be applied to evaluate and/or further the understanding of broader cytokine systems. The inventors demonstrated that by fine-tuning the receptor-JAK interactions, the expansive gap in potency between type I and III IFNs can be significantly narrowed, if not entirely eliminated in certain aspects. Ultimately, the findings in the present study have opened up future venues for continued research that will have significant impact not only on expanding the canonical understanding of the JAK/STAT pathway in the context of IFN signaling but also on devising novel strategies for clinical translations of type III IFNs.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

### REFERENCES

1. Isaacs, A. & Lindenmann, J. Virus interference. I. The interferon. Proc R Soc Lond B Biol Sci 147, 258-267, doi:10.1098/rspb. 1957.0048 (1957).
2. Vilček, J. & Feldmann, M. Historical review: Cytokines as therapeutics and targets of therapeutics. Trends in Pharmacological Sciences 25, 201-209, doi:https://doi.org/10.1016/j.tips.2004.02.011 (2004).
3. Lazear, H. M., Schoggins, J. W. & Diamond, M. S. Shared and Distinct Functions of Type I and Type III Interferons. Immunity 50, 907-923, doi:10.1016/j.immuni.2019.03.025 (2019).
4. de Weerd, N. A. & Nguyen, T. The interferons and their receptors—distribution and regulation. Immunology and cell biology 90, 483-491, doi:10.1038/icb.2012.9 (2012).
5. Zhang, J.-M. & An, J. Cytokines, inflammation, and pain. International anesthesiology clinics 45, 27-37, doi:10.1097/AIA.0b013e318034194e (2007).
6. Oppenheim, J. J. Cytokines: past, present, and future. Int J Hematol 74, 3-8, doi:10.1007/bf02982543 (2001).

7 Turner, M. D., Nedjai, B., Hurst, T. & Pennington, D. J. Cytokines and chemokines: At the crossroads of cell signalling and inflammatory disease. Biochimica et Biophysica Acta (BBA)—Molecular Cell Research 1843, 2563-2582, doi:https://doi.org/10.1016/j.bbamcr.2014.05.014 (2014).
8. Levy, D. E., Marie, I. J. & Durbin, J. E. Induction and function of type I and III interferon in response to viral infection. Current opinion in virology 1, 476-486, doi:10.1016/j.coviro.2011.11.001 (2011).
9 Lazear, H. M. et al. IRF-3, IRF-5, and IRF-7 coordinately regulate the type I IFN response in myeloid dendritic cells downstream of MAVS signaling. PLOS Pathog 9, e1003118, doi:10.1371/journal.ppat.1003118 (2013).
10. Osterlund, P. I., Pietilä, T. E., Veckman, V., Kotenko, S. V. & Julkunen, I. IFN regulatory factor family members differentially regulate the expression of type III IFN (IFN-lambda) genes. J Immunol 179, 3434-3442, doi:10.4049/jimmunol. 179.6.3434 (2007).
11. Kotenko, S. V. et al. IFN-lambdas mediate antiviral protection through a distinct class II cytokine receptor complex. Nat Immunol 4, 69-77, doi:10.1038/ni875 (2003).
12. Forero, A. et al. Differential Activation of the Transcription Factor IRF1 Underlies the Distinct Immune Responses Elicited by Type I and Type III Interferons. Immunity 51, 451-464.e456, doi:https://doi.org/10.1016/j.immuni.2019.07.007 (2019).
13. Piehler, J., Thomas, C., Garcia, K. C. & Schreiber, G. Structural and dynamic determinants of type I interferon receptor assembly and their functional interpretation. Immunological reviews 250, 317-334, doi:10.1111/imr.12001 (2012).
14. Mendoza, J. L. et al. The IFN-lambda-IFN-lambdaR1-IL-10Rbeta Complex Reveals Structural Features Underlying Type III IFN Functional Plasticity. Immunity 46, 379-392, doi:10.1016/j.immuni.2017.02.017 (2017).
15. Ait-Oufella, H., Taleb, S., Mallat, Z. & Tedgui, A. Recent Advances on the Role of Cytokines in Atherosclerosis. Arteriosclerosis, Thrombosis, and Vascular Biology 31, 969-979, doi:10.1161/ATVBAHA.110.207415 (2011).
16. Dinarello, C. A. Immunological and inflammatory functions of the interleukin-1 family. Annu Rev Immunol 27, 519-550, doi:10.1146/annurev.immunol.021908.132612 (2009).
17. Wang, X., Lupardus, P., Laporte, S. L. & Garcia, K. C. Structural biology of shared cytokine receptors. Annual review of immunology 27, 29-60, doi:10.1146/annurev.immunol.24.021605.090616 (2009).
18. Shaw, A. E. et al. Fundamental properties of the mammalian innate immune system revealed by multispecies comparison of type I interferon responses. PLOS Biol 15, e2004086, doi:10.1371/journal.pbio.2004086 (2017).
19. Crotta, S. et al. Type I and type III interferons drive redundant amplification loops to induce a transcriptional signature in influenza-infected airway epithelia. PLOS Pathog 9, e1003773, doi:10.1371/journal.ppat.1003773 (2013).
20. Zhou, Z. et al. Type III interferon (IFN) induces a type I IFN-like response in a restricted subset of cells through signaling pathways involving both the Jak-STAT pathway and the mitogen-activated protein kinases. J Virol 81, 7749-7758, doi:10.1128/jvi.02438-06 (2007).
21. Feld, J. J. et al. Peginterferon lambda for the treatment of outpatients with COVID-19: a phase 2, placebo-controlled randomised trial. Lancet Respir Med 9, 498-510, doi:10.1016/s2213-2600 (20) 30566-x (2021).
22. Janssen, H. L. et al. Pegylated interferon alfa-2b alone or in combination with lamivudine for HBeAg-positive chronic hepatitis B: a randomised trial. Lancet 365, 123-129, doi:10.1016/s0140-6736 (05) 17701-0 (2005).
23. Yao, J. C. et al. Phase III prospective randomized comparison trial of depot octreotide plus interferon alfa-2b versus depot octreotide plus bevacizumab in patients with advanced carcinoid tumors: SWOG S0518. Journal of Clinical Oncology 35, 1695 (2017).
24. Lau, G. K. et al. Peginterferon Alfa-2a, lamivudine, and the combination for HBeAg-positive chronic hepatitis B. N Engl J Med 352, 2682-2695, doi:10.1056/NEJMoa043470 (2005).
25. Jagannathan, P. et al. Peginterferon Lambda-1a for treatment of outpatients with uncomplicated COVID-19: a randomized placebo-controlled trial. Nature Communications 12, 1967, doi:10.1038/s41467-021-22177-1 (2021).
26. Lazear, H. M., Nice, T. J. & Diamond, M. S. Interferon-λ: Immune Functions at Barrier Surfaces and Beyond. Immunity 43, 15-28, doi:10.1016/j.immuni.2015.07.001 (2015).
27. O'Shea, J. J., Gadina, M. & Schreiber, R. D. Cytokine signaling in 2002: new surprises in the Jak/Stat pathway. Cell 109 Suppl, S121-131, doi:10.1016/s0092-8674(02) 00701-8 (2002).
28. McComb, S. et al. Type-I interferon signaling through ISGF3 complex is required for sustained Rip3 activation and necroptosis in macrophages. Proceedings of the National Academy of Sciences 111, E3206-E3213, doi:10.1073/pnas.1407068111 (2014).
29. Foster, J. R. The functions of cytokines and their uses in toxicology. International journal of experimental pathology 82, 171-192, doi:10.1046/j.1365-2613.2001.iep0082-0171-x (2001).
30. Bousoik, E. & Montazeri Aliabadi, H. “Do We Know Jack” About JAK? A Closer Look at JAK/STAT Signaling Pathway. Frontiers in Oncology 8, doi:10.3389/fonc.2018.00287 (2018).
31. Constantinescu, S. N., Huang, L. J.-s., Nam, H.-s. & Lodish, H. F. The Erythropoietin Receptor Cytosolic Juxtamembrane Domain Contains an Essential, Precisely Oriented, Hydrophobic Motif. Molecular Cell 7, 377-385, doi:https://doi.org/10.1016/S1097-2765 (01) 00185-X (2001).
32. Moraga, I. et al. Synthekines are surrogate cytokine and growth factor agonists that compel signaling through non-natural receptor dimers. eLife 6, e22882, doi:10.7554/eLife.22882 (2017).
33. Wack, A., Terczyńska-Dyla, E. & Hartmann, R. Guarding the frontiers: the biology of type III interferons. Nature Immunology 16, 802-809, doi:10.1038/ni.3212 (2015).
34. Mendoza, J. L. et al. The IFN-λ-IFN-λR1-IL-10Rβ Complex Reveals Structural Features Underlying Type III IFN Functional Plasticity. Immunity 46, 379-392, doi:10.1016/j.immuni.2017.02.017 (2017).
35. Pestka, S., Langer, J. A., Zoon, K. C. & Samuel, C. E. Interferons and their actions. Annu. Rev. Biochem 56, 727-777, doi:10.1146/annurev.bi.56.070187.003455 (1987).
36. Lee, A. J. & Ashkar, A. A. The Dual Nature of Type I and Type II Interferons. Frontiers in Immunology 9, doi:10.3389/fimmu.2018.02061 (2018).
37. Pestka, S., Krause, C. D. & Walter, M. R. Interferons, interferon-like cytokines, and their receptors. Immunol Rev 202, 8-32, doi:10.1111/j.0105-2896.2004.00204.x (2004).
38. O'Shea, J. J., Holland, S. M. & Staudt, L. M. JAKs and STATs in immunity, immunodeficiency, and cancer. N Engl J Med 368, 161-170, doi:10.1056/NEJMra1202117 (2013).
39. Morris, R., Kershaw, N. J. & Babon, J. J. The molecular details of cytokine signaling via the JAK/STAT pathway. Protein science: a publication of the Protein Society 27, 1984-2009, doi:10.1002/pro.3519 (2018).
40. Turkson, J. & Jove, R. STAT proteins: novel molecular targets for cancer drug discovery. Oncogene 19, 6613-6626, doi:10.1038/sj.onc. 1204086 (2000).
41. Ferrao, R. et al. The Structural Basis for Class II Cytokine Receptor Recognition by JAK1. Structure 24, 897-905, doi:10.1016/j.str.2016.03.023 (2016).
42. Zhang, D., Wlodawer, A. & Lubkowski, J. Crystal Structure of a Complex of the Intracellular Domain of Interferon λ Receptor 1 (IFNLR1) and the FERM/SH2 Domains of Human JAK1. J. Mol. Biol. 428, 4651-4668, doi:10.1016/j.jmb.2016.10.005 (2016).
43. Wallweber, H. J., Tam, C., Franke, Y., Starovasnik, M. A. & Lupardus, P. J. Structural basis of recognition of interferon-α receptor by tyrosine kinase 2. Nat Struct Mol Biol 21, 443-448, doi:10.1038/nsmb.2807 (2014).
44. Argetsinger, L. S. et al. Identification of JAK2 as a growth hormone receptor-associated tyrosine kinase. Cell 74, 237-244, doi:10.1016/0092-8674 (93) 90415-m (1993).
45. Shuai, K., Schindler, C., Prezioso, V. R. & Darnell, J. E., Jr. Activation of transcription by IFN-gamma: tyrosine phosphorylation of a 91-kD DNA binding protein. Science 258, 1808-1812, doi:10.1126/science. 1281555 (1992).
46. Schindler, C. & Darnell, J. E., Jr. Transcriptional responses to polypeptide ligands: the JAK-STAT pathway. Annu. Rev. Biochem 64, 621-651, doi:10.1146/annurev.bi.64.070195.003201 (1995).
47. Murray, P. J. Understanding and exploiting the endogenous interleukin-10/STAT3-mediated anti-inflammatory response. Curr. Opin. Pharmacol. 6, 379-386, doi:10.1016/j.coph.2006.01.010 (2006).
48. Irons Nicholas, J. & Raftery Adrian, E. Estimating SARS-COV-2 infections from deaths, confirmed cases, tests, and random surveys. Proceedings of the National Academy of Sciences 118, e2103272118, doi:10.1073/pnas.2103272118 (2021).
49. Manns, M. P. et al. Peginterferon alfa-2b plus ribavirin compared with interferon alfa-2b plus ribavirin for initial treatment of chronic hepatitis C: a randomised trial. Lancet 358, 958-965, doi:10.1016/s0140-6736 (01) 06102-5 (2001).
50. Kalil, A. C. et al. Efficacy of interferon beta-1a plus remdesivir compared with remdesivir alone in hospitalised adults with COVID-19: a double-bind, randomised, placebo-controlled, phase 3 trial. Lancet Respir Med 9, 1365-1376, doi:10.1016/s2213-2600 (21) 00384-2 (2021).
51. Lasfar, A. et al. Characterization of the Mouse IFN-2 Ligand-Receptor System: IFN-λs Exhibit Antitumor Activity against B16 Melanoma. Cancer Research 66, 4468-4477, doi:10.1158/0008-5472.can-05-3653 (2006).
52. Thomas, C. et al. Structural linkage between ligand discrimination and receptor activation by type I interferons. Cell 146, 621-632, doi:10.1016/j.cell.2011.06.048 (2011).

SEQUENCES

| | | SEQ |
|---|---|---|
| | | ID |
| Name | Sequence | NO: |
| IFN-λR1 | LLILLLVIAAGGVIWKTLM | 1 |
| transmembrane | | |
| domain | | |
| insertion | | |
| site | | |
| WT | | |
| AA1 | LLILLLVAIAAGGVIWKTLM | 2 |
| AA2 | LLILLLVAAIAAGGVIWKTLM | 3 |
| AA3 | LLILLLVAAAIAAGGVIWKTLM | 4 |
| AA4 | LLILLLVAAAAIAAGGVIWKTLM | 5 |
| ISG15_fwd | CGCAGATCACCCAGAAGATCG | 6 |
| ISG15_rev | TTCGTCGCATTTGTCCACCA | 7 |
| MX1_fwd | GTTTCCGAAGTGGACATCGCA | 8 |
| MX1_rev | CTGCACAGGTTGTTCTCAGC | 9 |
| APOL3_fwd | GGGACGAGTCTGGCCCTTA | 10 |
| APOL3_rev | TCAATCGGTCAATGCTGGTTG | 11 |
| SAMD9L_fwd | GAAACAGGAGCACTCAATCTCA | 12 |
| SAMD9L_rev | CAGCCTTACTGGTGATTTTCACA | 13 |
| 18S_fwd | GTAACCCGTTGAACCCCATT | 14 |
| 18S_rev | CCATCCAATCGGTAGTAGCG | 15 |

What is claimed is:

1. An engineered cytokine receptor, wherein the receptor has an altered geometry that potentiates signaling when the receptor is bound by a ligand.

2. A method of treating a patient for cancer and/or a viral infection, comprising:
a) administering to the patient a therapeutically effective amount of a therapeutic agent that potentiates signaling through a cytokine receptor complex to provide a therapeutic effect; and
b) treating the cancer and/or viral infection.

3. The method of claim 1, wherein the cancer is melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplastic syndrome, multiple myeloma, transitional cell carcinoma, neuroblastoma, plasma cell neoplasms, Wilm's tumor, glioblastoma, retinoblastoma, or hepatocellular carcinoma.

4. The method of claim 1, wherein the virus causing the viral infection is HBV, HBV/HDV co-infection, Norovirus, Influenza, and/or SARS-COV2.

5. An engineered cytokine receptor complex, comprising:
a) one or more cytokine receptors;
b) a transmembrane domain that comprises one or more mutations that promote heterodimerization of the receptor; and
c) optionally, one or more high-affinity ligands bound to the one or more cytokine receptors.

6. A cell, comprising the engineered cytokine receptor of claim 5.

7. The cell of claim 6, wherein the one or more cytokine receptors elicits signaling through a Janus kinase/Signal Transducer and Activator of Transcription (JAK/STAT) pathway in the cell.

8. The cell of any one of claims 6-7, wherein the engineered cytokine receptor is a Type III interferon receptor.

9. The cell of claim 8, wherein the engineered cytokine receptor is IFNλR1, IL10Rβ, or IFNλ3 H11.

10. The cell of claim 9, wherein the engineered cytokine receptor comprises one or more mutations compared to the corresponding wild type receptor.

11. The cell of claim 10, wherein the one or more mutations introduces at least one alanine into alpha-helical transmembrane domain of the receptor.

12. The cell of claim 10, wherein the one or more mutations renders the receptor able to heterodimerize through the transmembrane domain.

13. The cell of claim 12, wherein the one or more mutations renders the transmembrane able to structurally twist such that janus kinases associated with the transmembrane domain are oriented to permit cross phosphorylation and activation.

14. The cell of claim 13, wherein the rotation decreases the distance between the kinase domains of janus kinases within the signaling complex, facilitating a more efficient transphosphorylation that leads to enhanced biological activities for Type III IFNs.

15. The cell of any one of claims 6-14, wherein the cell is an immune cell.

16. The cell of claim 15, wherein the cell is in vitro.

17. The cell of claim 15, wherein the cell is in vivo.

18. The cell of claim 17, wherein the cell is in vivo in a mammal.

19. The cell of claim 18, wherein the mammal is a human.

20. The cell of any one of claims 17-19, wherein the human is in need of therapy and a therapeutically effective amount of cells is provided to the human.


SEQUENCE LISTING (15 sequences)

SEQ ID NO: 1 - protein, 19 aa, synthetic construct
LLILLLVIAAGGVIWKTLM

SEQ ID NO: 2 - protein, 20 aa, synthetic construct
LLILLLVAIAAGGVIWKTLM

SEQ ID NO: 3 - protein, 21 aa, synthetic construct
LLILLLVAAIAAGGVIWKTLM

SEQ ID NO: 4 - protein, 22 aa, synthetic construct
LLILLLVAAAIAAGGVIWKTLM

SEQ ID NO: 5 - protein, 23 aa, synthetic construct
LLILLLVAAAAIAAGGVIWKTLM

SEQ ID NO: 6 - DNA, 21 bp, synthetic construct
  source 1..21: mol_type = other DNA
cgcagatcacccagaagatcg

SEQ ID NO: 7 - DNA, 20 bp, synthetic construct
  source 1..20: mol_type = other DNA
ttcgtcgcatttgtccacca

SEQ ID NO: 8 - DNA, 21 bp, synthetic construct
  source 1..21: mol_type = other DNA
gtttccgaagtggacatcgca

SEQ ID NO: 9 - DNA, 20 bp, synthetic construct
  source 1..20: mol_type = other DNA
ctgcacaggttgttctcagc

SEQ ID NO: 10 - DNA, 19 bp, synthetic construct
  source 1..19: mol_type = other DNA
gggacgagtctggccctta

SEQ ID NO: 11 - DNA, 21 bp, synthetic construct
  source 1..21: mol_type = other DNA
tcaatcggtcaatgctggttg

SEQ ID NO: 12 - DNA, 22 bp, synthetic construct
  source 1..22: mol_type = other DNA
gaaacaggagcactcaatctca

SEQ ID NO: 13 - DNA, 23 bp, synthetic construct
  source 1..23: mol_type = other DNA
cagccttactggtgattttcaca

SEQ ID NO: 14 - DNA, 20 bp, synthetic construct
  source 1..20: mol_type = other DNA
gtaacccgttgaaccccatt

SEQ ID NO: 15 - DNA, 20 bp, synthetic construct
  source 1..20: mol_type = other DNA
ccatccaatcggtagtagcg